(19) United States
(12) Patent Application Publication
(10) Pub. No.: US 2007/0104719 A1
(43) Pub. Date: May 10, 2007

(54) Caspase Activated Prodrugs Therapy

(75) Inventors: Paul J. Carter, Mercer Island, WA (US); Lewis Gazzard, North Yorkshire (GB)

Correspondence Address: MERCHANT & GOULD PC, P.O. BOX 2903, MINNEAPOLIS, MN, 55402-0903, US

(73) Assignee: Genentech, Inc., South San Francisco, CA (US)

(21) Appl. No.: 11/537,578

(22) Filed: Sep. 29, 2006

Related U.S. Application Data
(63) Continuation of application No. 10/182,975, filed on Aug. 2, 2002.
(60) Provisional application No. 60/184,779, filed on Feb. 24, 2000.

(51) Int. Cl. A61K 39/395

(52) U.S. Cl. 424/155.1; 424/178.1

(57) ABSTRACT

The invention provides novel method for the localized delivery of pharmaceutical agents by the administration of a caspase conjugate that targets a cell type of interest and the additional administration of a pro-agent that is locally converted, in the presence of the caspase, to an active agent. The invention further provides novel tageting agents comprising a caspase as well as novel prodrugs comprising a caspase cleavable prodrug moiety. The invention also provides pharmaceutical compositions as well as methods of treatment comprising the caspase conjugates and prodrugs of the invention

### BACKGROUND OF THE INVENTION

1. Field of the Invention

This invention relates to novel methods for the localized delivery of pharmaceutical agents by the administration of a caspase conjugate that targets a cell type of interest and the additional administration of a pro-agent that is locally converted, in the presence of the caspase, to an active agent. In particular embodiments, the invention relates to the targeted administration of prodrugs, such as those useful in cancer therapies, to areas characterized by various cell types, such as neoplastic cells, and the local conversion of the prodrug to active drug by a caspase in the area of the particular cell type. The invention provides novel tageting agents comprising a caspase as well as novel prodrugs comprising a caspase cleavable prodrug moiety. The invention also relates to pharmaceutical compositions as well as methods of treatment comprising the caspase conjugates and prodrugs of the invention.

2. Description of Related Disclosures

The use of antibody conjugates for the local delivery of cytotoxic agents to tumor cells in the treatment of cancer has been described. (Syrigos and Epenetos (1999) Anticancer Research 19:605-614; Niculescu-Duvaz and Springer (1997) Adv. Drg Del. Rev. 26:151-172; U.S. Pat. No. 4,975,278). Local delivery of cytotoxic agents to tumors is desirable where systemic administration of these agents results in the killing of normal cells as well as the tumor cells sought to be eliminated. According to one antitumor drug delivery system, a cytotoxic agent is conjugated to a tumor-specific antibody to form an immunoconjugate that binds to the tumor cells and thereby “delivers” the cytotoxic agent to the site of the tumor. The immunoconjugates utilized in these targeting systems include antibody-drug conjugates (see, e.g., Baldwin et al., (1986) Lancet pp. (Mar. 15, 1986):603-05) and antibody-toxin conjugates (Thorpe, “Antibody Carriers Of Cytotoxic Agents In Cancer Therapy: A Review,” in Monoclonal Antibodies '84: Biological And Clinical Applications, A. Pinchera et al. (ed.s), pp. 475-506 (1985)). Both polyclonal antibodies and monoclonal antibodies have been reported as useful in these strategies (Rowland et al., (1986) Cancer Immunol. Immunother., 21:183-87). Drugs used in these methods include include daunomycin, doxorubicin, methotrexate and vindesine (Rowland et al., (1986) supra). Toxins used in the antibody-toxin conjugates include bacterial toxins such as diphtheria toxin, plant toxins such as ricin as well as small molecule toxins such as maytansinoids (Liu et al., (1996) Proc. Natl. Acad. Sci. USA 93:8618-8623) and calicheamicin (Lode et al., (1998) Cancer Res. 58:2928; Hinman et al., (1993) Cancer Res. 53:3336-3342).

ADEPT is a two-step approach to drug delivery in which an antibody-enzyme fusion protein or conjugate is administered to a subject followed by a prodrug (Syrigos and Epenetos (1999) supra; Niculescu-Duvaz and Springer (1997) supra). The antibody conjugate is allowed to localize to the tumor target. An inactive prodrug is administered once unbound fusion protein has been allowed to clear from the circulation. The prodrug is activated enzymatically within and around the tumor by the localized enzyme conjugate.

ADEPT has proven to be an effective anti-tumor strategy in murine xenograft models (Syrigos and Epenetos (1999) supra). However, bacterial enzymes commonly employed in ADEPT models as well as the rodent derived antibodies used in early clinical trials may be immunogenic in mammalian systems (Sharma (1992) Cell Biophysics 21:109-120). ADEPT using a humanized antibody-human β-glucuronidase fusion protein was efficacious in mice (Bosslet et al., (1994) Cancer Res. 54:2151-2159). However, because of its very large size (150 kDa) human β-glucuronidase is not a prefered enzyme for ADEPT. As well, the use of human enzymes in human systems poses risks of unwanted activation of prodrug by endogenous enzymes and interference from endogenous substrates or inhibitors. Human carboxypeptidase A1 has been engineered so that it will activate a prodrug that is not a substrate for the wild-type enzyme (Smith et al., (1997) J. Biol. Chem. 272:15804-15816). It was not effective in vivo (Wolfe et al., (1999) Bioconjugate Chemistry 10:38-48).

Caspases are a family of intracellular cysteine proteases with roles in cytokine maturation and apoptosis (Talamian, et al., (1997) J. Biol. Chem. 272:9677-9682). Caspases are produced as single chain zymogens requiring proteolysis for activation (Stennick and Salvesen (1998) Biochimica et Biophysica Acta 1378:17-31). Caspase 3 (previously known as Yama, apopain and CPP32) is a relatively small (57 kDa) mammalian protease. It cleaves after the sequences Asp-Glu-Val-Asp (SEQ ID NO:3) and Asp-Glu-Ile-Asp (SEQ ID NO:4), a substrate specificity shared only by other caspases such as caspase 7 (Thornberry et al., (1997) J. Biol. Chem. 272:17907-17911). Endogenous caspase 3 and 7 are very tightly regulated and believed to be active only in cells undergoing apoptosis.

The HER2/neu protooncogene (also known as c-erbB2) is amplified and/or overexpressed in 20-30% of primary human breast and ovarian cancers and is a strong prognosticator of decreased overall survival and time to relapse (Slamon et al., (1987) Science 235:177-182; Slamon et al., (1989) Science 244:707-712). Numerous antibody-based strategies have been developed as potential therapeutics for cancers which overexpress the p185^{HER2} product of the HER2 /neu gene (Shalaby et al., (1992) J. Exp. Med. 175:217-225; Baselga et al., (1996) J. Clin. Onc. 14:737-744; Pegram et al., J. Clin. Onc. (1998) 16:2659-2671).

The humanized anti-p185^{HER2} antibody, humAb4D5-8 (Herceptin) (Carter et al., (1992a) Proc. Natl. Acad. Sci. USA 89:4285-4289) has shown anti-tumor activity both as a single agent (Basegla et al., (1996) J. Clin. Onc. 14:737-744) and in combination with cytotoxic chemotherapy (Pegram et al., (1998) J. Clin. Onc. 16:2659-71) in phase II clinical trials for the treatment of metastatic breast cancer. Herceptin was approved by the Federal Drug Administration in September 1998 for the treatment of metastatic breast cancer following two pivotal phase III trials (Cobleigh et al., (1999) J. Clin. Onc. 17:2639-2648).

Herceptin has been used as a building block to design other potentially more potent immunotherapeutics. These include humanized bispecific F(ab′)2 and diabody fragments for the retargeting of cytotoxic T cells (Shalaby et al, (1992) J. Exp. Med. 172:217-225; Zhu et al., (1995) Intern. J. Cancer 62:319-324; Zhu et al., (1996) Bio/Technology 14:192-196) stealth immunoliposomes for targeted drug delivery (Park et al., (1995) Proc. Natl. Acad. Sci. USA 92:1327-1331), and a disulfide-stabilized Fv-β-lactamase fusion protein for prodrug activation (Rodrigues et al., (1995) Chemistry and Biology 2:223-227; Kirpotin (1997) Biochemistry 36:66-75).

### SUMMARY OF THE INVENTION

The present invention provides novel methods and compositions useful in the diagnosis, prognosis and treatment of variety of diseases or disorders. The invention includes methods for the localized delivery of pharmaceutical agents by the administration of a caspase conjugate that targets a cell type of interest and the additional administration of a pro-agent that is locally converted by the caspase, to an active agent. In particular embodiments, the invention provides a method for the delivery of a cytotoxic drug to a cell type of interest comprising the steps of administering an effective amount of a cell targeted caspase conjugate which converts a caspase convertable cytotoxic prodrug to an active cytotoxic drug and the administration of the caspase convertable prodrug.

The invention provides for compositions, especially pharmaceutical compositions comprising a caspase. In preferred embodiments, the caspase is provided as a targeted caspase conjugate. Caspase conjugates according to the present invention include caspase/targeting agent complexes, especially caspase-antibody conjugates wherein a constituitively active caspase is linked to a targeting agent such as an antibody either through chemical cross linking or recombinant fusion.

According to the invention, the caspase conjugate targets or homes to a cell type of interest. Therefore, according to the invention, a caspase is linked to a targeting agent, preferably by fusion or chemical conjugation. Preferred targeting agents include naturally occurring and engineered receptor ligands, peptide and peptidometic ligands, antibodies, especially monoclonal antibodies, including antibody fragments such as Fab, Fab′, F(ab′)2, and Fv fragments, diabodies, linear antibodies, single-chain antibody molecules, multispecific antibodies formed from antibody fragments and the like. Preferred among targeting agents are antibodies.

Preferred caspases according to the present invention are mammalian caspases, including any of human caspases 1-10, especially constituively active caspases such as reverse caspases. In preferred embodiments, the methods and compositions employ a proapoptotic constituitively active caspase. Preferred according to this aspect of the invention are caspases selected from the group consisting of caspase 2, caspase 3 and caspase 7 and preferably caspase 3.

The invention further provides for methods of treating various diseases or disorders especially those characterized by the appearance or presence of a particular cell type. Such cells include bacterially and virally infected cells expressing cell surface epitopes characteristic of the infection, neoplastic and malignant cells such as tumor cells and cells characterized by their presence or appearance in areas of inflammation. The invention provides a method of treating a disease or disorder comprising the step of administering to a subject in need thereof a caspase conjugate of the invention. In a particular embodiment the invention provides a method of treating a disease or disorder characterized by the expression of a neoplastic or malignant cell type utilizing an antibody that targets the neoplastic or malignant cell type. In preferred embodiments, the invention provides a method of treating a disease or disorder characterized by the presence of a cell type expressing, for example Apo2, CD20, CD40, muc-1, prostate specific membrane antigen (PSMA), prostate stem cell antigen (PSCA), epithelial growth factor receptor (EGFR), CD33, CD19, decay accelerating factor (DAF), EpCAM, CD52, carcinoembryonic antigen (CEA), TAG72 antigen, c-MET, six-transmembrane epithelial antigen of the prostate (STEAP) or ErbB2. According to particular aspects the methods comprise administration of caspase-antibody conjugates wherein the antibody is an anti-CD20, anti-CD40, anti-ErbB2 or anti-Apo2 antibody, especially a monoclonal antibody or antibody fragment.

The invention further provides a method of delivering an active agent such as a cytotoxic drug to a particular cell type comprising the step of administering a pro-agent that is converted to an active agent in the presence of a caspase. Suitable pro-agents comprise a caspase cleavable prodrug moiety such as an Asp-Xaa-Xaa-Asp, Asp-Glu-Xaa-Asp, Asp-Glu-Val-Asp (SEQ ID NO:3) or Asp-Glu-Ile-Asp (SEQ ID NO:4) peptide sequence. Preferred pro-agents include pro-cytotoxic agents. Preferred proagents within the context of the present invention include cytotoxic pro-agents selected from the group consisting of maytansinoids, calicheamicin, doxorubicin, daunorubicin, epirubicin, taxol, taxotere, vincristine, vinblastine, mitomycin C, etoposide, methotrexate, cisplatin, cyclophosphamide, melphalan, Halotestin, cyclophosphamide, Thio-TEPA, chlorambucil, 5-FU, and cytoxan wherein the pro-agent comprises a caspase cleavable prodrug moeity.

The invention includes compositions, including pharmaceutical compositions comprising pro-agents and targeted caspase conjugates such as caspase-antibody fusion proteins for the treatment of a variety of diseases or disorders as well as kits and articles of manufacture. Kits and articles of manufacture preferably include:
(a) a container;
(b) a label on said container; and
(c) a composition comprising a targeted caspase conjugate contained within said container; wherein the composition is effective for treating a disease or disorder, the optional label on said container indicates that the composition can be used for treating a particular disease or disorder. The kits optionally include other components such as a caspase activatable prodrug or agent as well as accessory components such as a container comprising a pharmaceutically-acceptable buffer and instructions for using the composition to treat a disease disorder.

### BRIEF DESCRIPTION OF THE DRAWINGS

FIG. 1. Cellular accumulation of caspase cleavable prodrug Ac-DEVD-PABC-Doxorubicin in SK-BR-3 and MCF7 cells. Uptake of doxorubicin was estimated from a standard curve prepared using known quantities of doxorubicin that were added to the previously untreated cells.

FIG. 2. In vitro cytotoxicity of caspase cleavable prodrug Ac-DEVD-PABC-Doxorubicin on SK-BR-3 and MCF7 breast carcinoma cells cells plus or minus caspase 3.

FIG. 3. In vitro cytotoxicity of Ac-DEVD-PABC-Doxorubicin in human lung carcinoma cells (H460) and colon carcinoma cells (HCT116).

FIG. 4. In vitro cytotoxicity of Ac-DEVD-PABC-Taxol in human lung carcinoma cells (H460) and colon carcinoma cells (HCT116).

FIG. 5. Stability of caspase 3 in human plasma.

FIG. 6. Nucleic acid (SEQ ID NO:1) and amino acid (SEQ ID NOs: 2 and 25) sequence of anti-HER2 Fab reverse caspase 3 conjugate in plasmid pLCrC3.HCrC3. SEQ ID NO:2 is encoded by nucleotide 439 to 1977 of SEQ ID NO:1. SEQ ID NO: 25,is encoded by nucleotide 2025 to 3605 of SEQ ID NO:1.

FIG. 7. Schematic representation of anti-HER2 Fab reverse caspase 3 conjugate pLCrC3.HCrC3 together with plasmids pLCr3 and pHCrC3 used in its construction.

FIG. 8. Preparation of Ac-DEVD-doxorubicin prodrug: (i) doxorubicin hydrochloride, DCC, HOSu, DIPEA, DMF, 0-23° C. and (ii) Pd(PPh₃)₄, Bu₃ SnH, AcOH, DMF, 23° C.

FIG. 9. Preparation of Ac-DEVD-PABC (Asp-Glu-Val-Asp-para amninobenzyloxycarbonyl) prodrug moiety. In this example DEVD is the caspase cleavable prodrug moiety and PABC is the self-immolative linker: (iii) 4-Aminobenzyl alcohol, EEDQ, DMF, 23° C. and (iv) 4-Nitrophenyl chloroformate, 2,6-lutidine, DCM, DMF, 23° C.

FIG. 10. Preparation of Ac-DEVD-PABC-doxorubicin prodrug: (v) doxorubicin hydrochloride, DIPEA, DMF, 23° C. and (vi) Pd(PPh₃)₄, Bu₃SnH, AcOH, DMF, 23° C.

FIG. 11. Preparation of Ac-DEVD-PABC-paclitaxel prodrug: (vii) Paclitexel, DMAP, MeCN, 23° C. and (viii) Pd(PPh₃)₄, Bu₃SnH, AcOH, DMF, 23° C.

### DETAILED DESCRIPTION OF THE PREFERRED EMBODIMENTS

Definitions

The term “amino acid” within the scope of the present invention is used in its broadest sense and is meant to include naturally occurring L-amino acids or residues. The commonly used one and three letter abbreviations for naturally occurring amino acids are used herein (Lehninger, A. L., Biochemistry, 2d ed., pp. 71-92, (1975), Worth Publishers, New York). The term includes D-amino acids as well as chemically modified amino acids such as amino acid analogs, naturally occurring amino acids that are not usually incorporated into proteins such as norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio (The Peptides: Analysis, Synthesis, Biology,) Eds. Gross and Meiehofer, Vol. 5 p 341, Academic Press, Inc, N.Y. 1983, which is incorporated herein by reference.

The terms antibody and immunoglobulin are used interchangeably and used to denote glycoproteins having certain structural characteristics. The term “antibody” is used in the broadest sense and specifically covers single monoclonal antibodies (including agonist and antagonist antibodies) and antibody compositions with polyepitopic specificity. The term “antibody” specifically covers monoclonal antibodies (including full length monoclonal antibodies), polyclonal antibodies, multispecific antibodies (e.g., bispecific antibodies), and antibody fragments.

In defining an antibody or immunoglobulin reference is made to immunoglobulins in general and in particular to the domain structure of immunoglobulins as applied to human IgG1 by Kabat E. A. (1978) Adv. Protein Chem. 32:1-75. Accordingly, immunoglobulins are generally heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has an amino terminal variable domain (VH) followed by carboxy terminal constant domains. Each light chain has a variable N-terminal domain (VL) and a C terminal constant domain; the constant domain of the light chain is aligned with the first constant domain (CH1) of the heavy chain, and the light chain variable domain is aligned with the variable domain of the heavy chain. According to the domain definition of immunoglobulin polypeptide chains, light (L) chains have two conformationally similar domains VL and CL; and heavy chains have four domains (VH, CH1, CH2, and CH3) each of which has one intrachain disulfide bridge.

Depending on the amino acid sequence of the constant (C) domain of the heavy chains, immunoglobulins can be assigned to different classes. There are five major classes of immunoglobulins: IgA, IgD, IgE, IgG, and IgM. The heavy-chain constant domains that correspond to the different classes of immunoglobulins are called α, δ, ε, γ, and μ domains respectively. Sequence studies have shown that the β chain of IgM contains five domains VH, CHμ1, CHμ2, CHμ3, and CHμ4. The heavy chain of IgE (ε) also contains five domains while the heavy chain of IgA (α) has four domains. The immunoglobulin class can be further divided into subclasses (isotypes), e.g., IgG1, IgG2, IgG3, IgG4, IgA1, and IgA2.

The subunit structures and three-dimensional configurations of different classes of immunoglobulins are well known. Of these IgA and IgM are polymeric and each subunit contains two light and two heavy chains. The heavy chain of IgG (γ) contains a length of polypeptide chain lying between the CH 1 and CH 2 domains known as the hinge region. The a chain of IgA has a hinge region containing an O-linked glycosylation site and the μ and ε chains do not have a sequence analogous to the hinge region of the γ and α chains, however, they contain a fourth constant domain lacking in the others. The domain composition of immunoglobulin chains can be summarized as follows:

Light Chain λ=Vλ Cλ
κ=Vκ Cκ

Heavy Chain IgG (γ)=VH CHγ1, hinge CHγ2 CHγ
IgM (μ)=VH CHμ1 CHμ2 CHμ³ CHμ4
IgA (α)=VH CHα1 hinge CHα2 CHα3
IgE (ε)=VH CHε1 CHε2 CHε3 CHε4
IgD (δ)=VH CHδ1 hinge CHδ2 CHδ3

“Hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

Papain digestion of antibodies produces two identical antigen-binding fragments, called “Fab” fragments, each with a single antigen-binding site, and a residual “Fe” fragment, whose name reflects its ability to crystallize readily. Pepsin treatment yields an F(ab′)2 fragment that has two antigen-combining sites and is still capable of cross-linking antigen.

The Fab fragment also contains the constant domain of the λ light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” is the minimum antibody fragment which contains a complete antigen-recognition and -binding site. This region consists of a dimer of one heavy chain and one light chain variable domain in tight, non-covalent association.

“Antibody fragments” comprise a portion of a full length antibody, generally the antigen binding or variable domain thereof. Examples of antibody fragments include Fab, Fab′, F(ab′)2, and Fv fragments; diabodies; linear antibodies; single-chain antibody molecules; and multispecific antibodies formed from antibody fragments.

“Single-chain Fv” or “sFv” antibody fragments comprise the VH and VL domains of antibody, wherein these domains are present in a single polypeptide chain. Generally, the Fv polypeptide further comprises a polypeptide linker between the VH and VL domains which enables the sFv to form the desired structure for antigen binding. For a review of sFv see Plückthun in The Pharmacology of Monoclonal Antibodies, vol. 113, Rosenburg and Moore eds. Springer-Verlag, New York, pp. 269-315 (1994).

The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The expression “linear antibodies” when used throughout this application refers to the antibodies described in Zapata et al. Protein Eng. 8(10):1057-1062 (1995). Briefly, these antibodies comprise a pair of tandem Fd segments (VH-CH1-VH-CH1) which form a pair of antigen binding regions. Linear antibodies can be bispecific or monospecific.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, liver cancer, prostate cancer, vulval cancer, thyroid cancer, hepatic carcinoma and various types of head and neck cancer.

A “caspase” according to the present invention is any member of the structurally related group of cysteine proteases that share a dominant primary specificity for cleaving peptide bonds following Asp residues (Stennicke, H. and Salvesen, G. (1998) Biochimica et Biophysica Acta 1387:17-31) and includes naturally occurring caspases as well as variants thereof as more fully described herein. A series of naturally occurring caspases are known to be produced (Stennicke and Salvesen (1998) supra). Amino acid sequences of the members of this series are not entirely homologous. However, the caspases in this series exhibit the same or similar type of proteolytic activity. In general, caspases share the following characteristics: i) they are homologous cysteine proteases belonging to the family C14 in the Barrett and Rawlings classification (Barrett, A. J., (1997) Eur. J. Biochem. 250:1-6); they cleave preferentially after Asp residues in a peptide substrate; they are present in the cytosol of animal cells; they contain a conserved QACXG (SEQ ID NO:5), where X is Arg, Gln or Gly, pentapeptide active site motif.

Caspases in general require Asp in the “P1” substrate position as that term is defined by Schecter, I., and Berger, A., (1967) Biochem. Biophys. Res. Commun. 27:157-162. Caspases have a specificity for peptide substrates and the primary sequence of the substrate is necessary for caspase enzymatic cleavage. The caspases can be divided in to three groups. Group I caspases (caspases 1, 4 and 5) all favor hydrophobic amino acids in the P4 positions with an optimal sequence Trp-Glu-His-Asp (SEQ ID NO:6) (P4-P3-P2-P1). Group II caspases (caspases 2, 3, 7 and CED-3) have a strict requirement for Asp in P4, preferring the sequence Asp-Glu-X-Asp. Group III caspases (caspases 6, 8, 9 and 10) tolerate many amino acids in P4 but have a preference for those with branched aliphatic sidechains and an optimal sequence of Val/Leu-Glu-X-Asp. All caspases prefer Glu as P3 Group I caspases are often termed mediators of inflammation, Group I caspases, effector of apoptosis and Group III activators or apoptosis.

TABLE I

Reference can be made to Thornberry et al.,
(1997) J. Biol. Chem. 272:17907-17911 as
well as the following:

| | |
|---|---|
| Caspase Group | Optimal Sequence |

| | | |
|---|---|---|
| Group I | | |
| Caspase 1 | WEHD | (SEQ ID NO:6) |
| Caspase 4 | W/LEHD | |
| Caspase 5 | W/LEHD | |
| Group II | | |
| Caspase 3 | DEVD | (SEQ ID NO:3) |
| Caspase 7 | DEVD | (SEQ ID NO:3) |
| Caspase 2 | DEHD | (SEQ ID NO:8) |
| CED-3 | DETD | (SEQ ID NO:9) |
| Group III | | |
| Caspase 6 | VEHD | (SEQ ID NO: 10) |
| Caspase 8 | LETD | (SEQ ID NO:1 1) |
| Caspase 9 | LEHD | (SEQ ID NO:7) |
| Caspase 10 | LE(Nle)D | (SEQ ID NO: 12) |

According to the present invention, caspases of Group II and III are referred to as “proapoptotic caspases.”

The term “caspase” and “wild type caspase” are used to refer to a polypeptide having an amino acid sequence corresponding to a naturally occurring caspase or recombinantly produced caspase having an amino acid sequence of a naturally occurring caspase. Naturally occurring caspases include those of human species as well as other animal species such as rabbit, rat, porcine, non human primate, equine, murine, and ovine. The amino acid sequence of the mammalian caspase proteins are generally known or obtainable through conventional techniques (Stennicke and Salvesen (1998) supra). Caspase amino acid sequences for caspases 1-10 as well as the number given to the amino acids are those described by Cohen, (1997) Biochem. J. 326:1-16.

“Caspase variant” and the like refer to caspase-type proteases having a sequence which is not found in nature but that is derived from or derivable from a precursor wild-type caspase. The caspase variant has the same substrate specificity as the precursor caspase but differs by virtue of amino acid substitutions within the wild type caspase amino acid sequence. Therefore caspase according to the instant invention is meant to include caspase variants in which the DNA sequence encoding the precursor caspase is modified to produce a mutant DNA sequence which encodes the substitution of one or more amino acids in the naturally occurring caspase amino acid sequence so long as the caspase meets activity and structure limitations described herein.

A “caspase convertable pro-agent” or “pro-agent” or “prodrug” within the context of the present invention refers to an agent such as a chemotherapeutic agent that requires enzymatic cleavage by a caspase for optimal activity and comprises a “caspase cleavable prodrug moiety” or “prodrug moiety” such as the peptidyl moieties listed above as caspase substrates. Proagents are generally 10 fold less active than the parent agent. In preferred embodiments the proagent is 10-100 fold less active than the parent agent. In further preferred embodiments the proagent is greater than 100 fold less active than the parent agent and more preferably greater than 1000 fold less active than the parent agent.

A caspase conjugate of the present invention will “target” a particular cell type if the target molecule binds the particular cell type with sufficient affinity and specificity to “home” to, “binds” or “targets” a specific cell type in vitro and preferably in vivo (see, for example, the use of the terms “homes to,” “homing,” and “targets” in Pasqualini and Ruoslahti (1996) Nature, 380:364-366 and Arap et al., (1998) Science 279:377-380). In general, the targeting molecule will bind a particular cell type or surface molecule thereon with an affinity of less than about 1 μM, preferably less about 100 nM and more preferably less than about 10 nM. However, targeting molecules having an affinity for a cellular epitope of less than about 1 nM and preferably between about 1 pM and 1 nM are equally likely to be targeting molecules within the context of the present invention.

As used within the context of the present invention the term “targeting molecule” or “agent” includes, proteins, peptides, glycoproteins such an antibodies, glycopeptides, glycolipids, polysaccharides, oligosaccharides, nucleic acids, and the like which bind to or are a ligand for a particular cellular epitope. Targeting agents according to the present invention include ligands such as antibodies, for cell associated molecules such as cellular receptors or cellular distribution (CD) antigens expressed on particular cell types, and include, for example:

i) ligands for organ selective address molecules on endothelial cell surfaces such as those which have been identified for lymphocyte homing to various lymphoid organs and to tissues undergoing inflammation (Belivaqua, et al (1989) Science, 243:1160-1165; Siegelman et al., (1989) Science 243:1165-1171; Cepek et al. (1994) Nature 372:190-193 and Rosen and Bertozzi (1994) Curr. Opin. Cell Biol. 6:663-673).

ii) ligands for endothelial cell markers such as Erb2 responsible for tumor homing to various organs (Johnson et al., (1993) J. Cell. Biol. 121:1423-1432) including “Heregulin” (HRG) which when used herein refers to a polypeptide encoded by the heregulin gene product as disclosed in U.S. Pat. No. 5,641,869 or Marchionni et al., Nature, 362:312-318 (1993). Examples of heregulins include heregulin-α, heregulin-β1, heregulin-β2 and heregulin-β3 (Holmes et al., Science, 256:1205-1210 (1992); and U.S. Pat. No. 5,641,869); neu differentiation factor (NDF) (Peles et al. Cell 69: 205-216 (1992)); acetylcholine receptor-inducing activity (ARIA) (Falls et al. Cell 72:801-815 (1993)); glial growth factors (GGFs) (Marchionni et al., Nature, 362:312-318 (1993)); sensory and motor neuron derived factor (SMDF) (Ho et al. J. Biol. Chem. 270:14523-14532 (1995)); -heregulin (Schaefer et al. Oncogene 15:1385-1394 (1997)). The term includes biologically active fragments and/or amino acid sequence variants of a native sequence HRG polypeptide, such as an EGF-like domain fragment thereof (e.g. HRG 1177-244).

iii) tumor cell antigens or “tumor antigens” that serve as markers for the presence of a preneoplastic or a neoplastic cell.

Examples of peptide type targeting molecules agents or ligands include, for example:

i) peptides capable of mediating selective localization to various organs such as brain and kidney (Pasqualini and Ruoslohti (1996) Nature 380:364-366). Often these peptides contain dominant amino acid motifs such as the Ser-Arg-Leu motif found in peptides localizing to brain (Pasqualini and Ruoslahti (1996) supra).

ii) peptides containing amino acid sequences recognizing structurally related receptors such as integrins. For example, the amino acid sequence Arg-Gly-Asp (RGD) is found in extracellular matrix proteins such as fibrinogen, fibronectin, von Willibrand Factor and thrombospondin that are known to bind various integrins found on platelets, endothelial cells leukocytes, lymphocytes, monocytes and granulocytes. Peptides containing the RGD motif can be used to modulate the activity of the RGD recognizing integrins (Gurrath et al., (1992) Eur. J. Biochem. 210:911-921; Koivunen et al., (1995) Bio/Technology 13:265-270; O'Neil et al., (1992) Proteins 14:509-515). For example, peptides capable of homing specifically to tumor blood vessels such as those identified by in vivo phage selection contain the Arg-Gly-Asp (RGD) motif embedded in the peptide structure and binds selectively to v 3 and v 5 integrins (Arap et al., (1998) Science 279:377-380).

iii) phage display of peptide libraries has yielded short peptides with well defined solution conformation that can bind, for example, insulin like growth factor binding protein-1 and produce insulin growth factor like activity (Lowman et al., (1998) Biochemistry 37:8870-8878.

iv) small peptides isolated by random phage disply of peptide libraries which bind to and activate the cellular receptors such as the receptor for EPO, optionally including full agonist peptides such as those which stimulate crythropoiesis described by Wrighton et al., (1996) Science 273:458-463; or those that stimulate proliferation of TPO responsive cells and described by Cwirla et al., (1997) Science 276:1696-1699).

By “ErbB ligand” is meant a polypeptide which binds to and/or activates an ErbB receptor, The ErbB ligand of particular interest herein is a native sequence human ErbB ligand such as epidermal growth factor (EGF) (Savage et al., J. Biol. Chem. 247:7612-7621 (1972)); transforming growth factor alpha (TGF-α) (Marquardt et al., Science 223:1079-1082 (1984)); amphiregulin also known as schwanoma or keratinocyte autocrine growth factor (Shoyab et al. Science 243:1074-1076 (1989); Kimura et al. Nature 348:257-260 (1990); and Cook et al. Mol. Cell. Biol. 11:2547-2557 (1991)); betacellulin (Shing et al., Science 259:1604-1607 (1993); and Sasada et al. Biochem. Biophys. Res. Commun. 190:1173 (1993)); heparin-binding epidermal growth factor (HB-EGF) (Higashiyama et al., Science 251:936-939 (1991)); epiregulin (Toyoda et al., J. Biol. Chem. 270:7495-7500 (1995); and Komurasaki et al. Oncogene 15:2841-2848 (1997)), a heregulin (see below); neuregulin-2 (NRG-2) (Carraway et al., Nature 387:512-516 (1997)); neuregulin-3 (NRG-3) (Zhang et al., Proc. Natl. Acad. Sci. 94:9562-9567 (1997)); or cripto (CR-1) (Kannan et al. J. Biol. Chem. 272(6):3330-3335 (1997)). ErbB ligands which bind EGFR include EGF, TGF-α, amphiregulin, betacellulin, HB-EGF and epiregulin. ErbB ligands which bind ErbB3 include heregulins. ErbB ligands capable of binding ErbB4 include betacellulin, epiregulin, HB-EGF, NRG-2, NRG-3 and heregulins.

Preferred targeting agents include naturally occurring and engineered receptor ligands, peptide and peptidometic ligands, antibodies, especially monoclonal antibodies, including antibody fragments such as Fab, Fab′, F(ab′)2, and Fv fragments, diabodies, linear antibodies, single-chain antibody molecules, multispecific antibodies formed from antibody fragments and the like. Preferred among targeting agents are antibodies.

A “chemotherapeutic agent” is a chemical compound useful in the treatment of cancer. Examples of chemotherapeutic agents include Maytansinoids such as Maytansine and Ansamitocins, as well as synthetic analogs thereof, the Enediyne antibiotics including; Calicheamicins, in particular Calicheamicin γ₁^{I} and Calicheamicin θ₁^{I} (see, Angew, (1994) Chem. Int. Ed. Engl., 33:183-186), Dynemicins, in particular Dynemicin A and synthetic analogs thereof and Neocarzinostatin chromophore and related Chromoprotein enediyne antibiotic chromophores, Esperamicins (see U.S. Pat. No. 4,675,187) such as Esperamicin A₁; Adriamycin (Doxorubicin) and Morpholino-doxorubicin (Morpholino-ADR), Cyanomorpholino-doxorubicin (Cyanomorpholino-ADR), 2-Pyrrolino-Doxorubicin also known as AN-201, Deoxydoxorubicin, Tichothecenes, in particular T-2 Toxin, Verracurin A, Roridin A and Anguidine, Epothilones, Rhizoxin, Acetogenins, in particular Bullatacin and Bullatacinone, Cryptophycins, in particular Cryptophycin 1 and Cryptophycin 8, Dolastatin, Callystatin, CC-1065 and synthetic analogs, in particular Adozelesin, Carzelesin and Bizelesin, Duocarmycins and synthetic analogs, in particular KW-2189 and CBI-TMI, Sarcodictyins, Eleutherobin, Spongistatins, Bryostatins, Pancratistatin, Camptothecin and synthetic analogs, in particular Topotecan, Epirubicin, 5-Fluorouracil, Cytosine Arabinoside (“Ara-C”), Cyclophosphamide, Thiotepa, Busulfan, Taxoids, e.g. Paclitaxel (TAXOL®, Bristol-Myers Squibb Oncology, Princeton, N.J.) and Docetaxel (Taxotere, Rhône-Poulenc Rorer, Antony, Rnace), Methotrexate, Cisplatin, Melphalan and other related nitrogen mustards, Vinblastine, Bleomycin, Etoposide, Ifosfamide, Mitomycins such as Mitomycin C, Mitoxantrone, Vincristine, Vinorelbine, Carboplatin, Teniposide, Daunomycin, Carminomycin, Aminopterin, Dactinomycin. Also included in this definition are hormonal agents that act to regulate or inhibit hormone action on tumors such as tamoxifen and onapristone.

The “CD20” antigen is expressed during early pre-B cell development and may regulate a step in cellular activation required for cell cycle initiation and differentiation. The CD20 antigen is expressed at high levels on neoplastic B cells; however, it is present on normal B cells as well. Anti-CD20 antibodies which recognize the CD20 surface antigen have been used clinically to lead to the targeting and destruction of neoplastic B cells (Maloney et al., (1994) Blood 84:2457-2466; Press et al., (1993) NEJM 329:1219-1224; Kaminski et al., (1993) NEJM 329:459-465; McLaughlin et al., (1996) Proc. Am. Soc. Clin. Oncol. 15:417). Chimeric and humanized anti-CD20 antibodies mediate complement dependent lysis of target B cells (Maloney et al. supra). The monoclonal antibody C2B8 recognizes the human B cell restricted differentiation antigen Bp35 (Liu et al., (1987) J. Immunol. 139:3521; Maloney et al., (1994) Blood 84:2457). “C2B8” is defined as the anti-CD20 monoclonal antibody described in International Publication No. WO94/11026.

A “disease” or “disorder” is any condition that would benefit from treatment with the compositions comprising the caspase conjugates and pro-agents of the invention. This includes chronic and acute disorders or diseases including those pathological conditions which predispose the mammal to the disorder in question. Non-limiting examples of disorders to be treated herein include benign and malignant tumors; leukemias and lymphoid malignancies; neuronal, glial, astrocytal, hypothalamic and other glandular, macrophagal, epithelial, stromal and blastocoelic disorders; and inflammatory, angiogenic and immunologic disorders.

The terms “HER2”, “ErbB2” “c-Erb-B2” are used interchangeably. Unless indicated otherwise, the terms “ErbB2” “c-Erb-B2” and “HER2” when used herein refer to the human protein and “her2”, “erbB2” and “c-erb-B2” refer to the human gene. The human erbB2 gene and ErbB2 protein are described in, for example, Semba et al., (1985) PNAS (USA) 82:6497-6501 and Yamamoto et al. (1986) Nature 319:230-234 (Genebank accession number X03363). ErbB2 comprises four domains (Domains 1-4).

The terms “agent” “pharmaceutical agent,” “drug,” “medicament” and the like are used interchangeably herein with the term “parent agent” or “parent drug” to refer to a compound, having some utility within the pharmacological sciences. The pharmaceutical agent is pharmaceutically active or “bioactive,” by virtue of possessing a biological activity such as cellular cytotoxicity in the absence of the caspase cleavable prodrug moiety of the present invention. Such molecules include small bioorganic molecules, e.g. peptidomimetics, antibodies, immunoadhesins, proteins, peptides, glycoproteins, glycopeptides, glycolipids, polysaccharides, oligosaccharides, nucleic acids, bioorganic molecules, pharmacological agents and their metabolites, transcriptional and translation control sequences, and the like.

“Procaspase” refers to a caspase sequence of inactive or minimally active zymogen where cleavage of an internal portion of the procaspase results in the appearance of the “mature” form of the caspase having substantially greater activity. Caspases are synthesized as zymogen the active forms consisting of a large (˜17-20 kDa) and a small (9-12 kDa) subunit, released from the precursor by proteolytic cleavage. Many proteolytic enzymes are found in nature as translational proenzyme products and, in the absence of post-translational processing, are expressed in this fashion.

The term “prodrug” is used herein to refer to a derivative of a parent drug that optionally has enhanced pharmaceutically desirable characteristics or properties (e.g. relative inactivity, transport, bioavailablity, pharmacodynamics, etc.) and requires “bioconversion,” i.e., cleavage of the “prodrug moiety” enzymatically by a caspase, to release the active parent drug.

Substrates are described in triplet or single letter code as Pn . . . P2-P1-P1′-P2′ . . . Pn′. The “P1 ” residue refers to the position proceeding (i.e., N-terminal to) the scissile peptide bond (i.e. between the P1 and P1′ residues) of the substrate as defined by Schechter and Berger (Schechter, I. and Berger, A., Biochem. Biophys. Res. Commun. 27: 157-162 (1967)). Similarly, the term P1′ is used to refer to the position following (i.e., C-terminal to) the scissile peptide bond of the substrate. Increasing numbers refer to the next consecutive position preceding (e.g., P2 and P3) and following (e.g., P2′ and P3′) the scissile bond. According to the present invention the scissile peptide bond is that bond that is cleaved by the caspases of the instant invention.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

The terms “treating,” “treatment,” and “therapy” as used herein refer include curative therapy, prophylactic therapy, and preventative therapy.

The term “mammal” as used herein refers to any mammal classified as a mammal, including humans, cows, sheep, horses, dogs and cats. In a preferred embodiment of the invention, the mammal is a human.

### MODES FOR CARRYING OUT THE INVENTION

The present invention relates to the targeted administration of caspases for the cleavage of caspase cleavable prodrugs and methods for the localized delivery of pharmaceutical agents by the administration of a caspase conjugate that targets a cell type of interest and the additional administration of a pro-agent that is locally converted, in the presence of the caspase, to an active agent. For ADEPT methods in general reference can be made to Syrigos and Epenetos (1999) supra. In particular embodiments the invention relates to the targeted administration of prodrugs, such as those useful in cancer therapies, to areas characterized by various cell types such as neoplastic cells and the local conversion of the prodrug to active drug by a caspase in the area of the particular cell type. The invention provides novel tageting agents comprising a caspase as well as novel prodrugs comprising a caspase cleavable prodrug moiety.

The caspase component of the present invention includes any caspase as defined herein. Preferably any of human caspases 1-10 or granzyme B. Preferred caspases are the proapoptotic caspases 2, 3, 6, 7, 8, 9, 10. Most preferred caspases are caspases 2, 3, 7

Caspases are attractive for prodrug activation as they have exquisite substrate specificity (Xaa-Glu-Xaa-Asp) which is unlike that of other known proteases aside from granzyme B. Proapoptotic caspases are widely distributed as inactive or minimally active zymogens but active enzymes are restricted to the intracellular compartments of cells undergoing apoptosis. The most favorable substrates for caspases 2, 3 and 7 are DEHD (SEQ ID NO:8), DEVD (SEQ ID NO:3) and DEVD (SEQ ID NO:3) respectively. These sequences are very poor substrates for granzyme B which has the preferred substrate IEPD (SEQ ID NO:13) (Thornberry et al., (1997) supra) and for proinflammatory caspases (caspases 1, 4, 5, 11, 12, 13) which have a preference for a large hydrophobic residue at S4 (caspase 1 WEHD (SEQ ID NO:6), caspase 4 (W/L)EHD, caspase 5 (W/L)EHD). For example Ac-DEVD-pna was found to readily hydrolyzed by recombinant commercial caspase 3 but there was no detectable cleavage by granzyme B.

Therefore according to the present invention, a caspase is selected to link to a particular targeting molecule, i.e. a molecule that will home to or bind a cell type of interest. The corresponding prodrug is constructed so that the inactive or prodrug form of the agent comprises a caspase cleavable moiety such as the peptidyl prodrug moieties described herein.

Since caspases are naturally occurring as zymogens it is necessary to generate constituitively active caspases. A convenient method for producing a constituitively active caspase is described in Srinivasula et al., (1998) J. Biological Chem. 273(17):10107-10111. According to this method caspases designated “reverse caspases” are generated by switching the order of the large and small subunits such that the engineered molecule mimics a structure presented by the processed wild type active molecule. While the foregoing provides a convenient method for producing an active caspase it is provided by way of exemplication and not limitation.

Targeting Component

The targeting component can be any molecule as described herein which binds to or homes to a cell type of interest. Antibody and peptide type molecules are preferred targeting molecules.

In preferred embodiments the targeting molecule is an antibody. The antibody component of the conjugate of the invention includes any antibody which binds specifically to particular cell type. For example, the antibody may bind a tumor-associated antigen. Examples of such antibodies include, but are not limited to, those which bind specifically to antigens found on carcinomas, melanomas, lymphomas and bone and soft tissue sarcomas as well as other tumors. Antibodies that remain bound to the cell surface for extended periods or that are internalized very slowly are preferred. These antibodies may be polyclonal or preferably, monoclonal, may be intact antibody molecules or fragments containing the active binding region of the antibody, e.g., Fab or F(ab′)2, and can be produced using techniques well established in the art.

Exemplary antibodies within the scope of the present invention include but are not limited to anti-IL-8, St John et al., (1993) Chest 103:932 and International Publication No. WO 95/23865; anti-CD11a, Filcher et al., Blood, 77:249-256, Steppe et al., (1991) Transplant Intl. 4:3-7, and Hourmant et al., (1994) Transplantation 58:377-380; anti-IgE, Presta et al., (1993) J. Immunol. 151:2623-2632, and International Publication No. WO 95/19181; anti-HER2, Carter et al., (1992) Proc. Natl. Acad. Sci. USA 89:4285-4289, and International Publication No. WO 92/20798; anti-VEGF, Jin Kim et al., (1992) Growth Factors, 7:53-64, and International Publication No. WO 96/30046; and anti-CD20, Maloney et al., (1994) Blood, 84:2457-2466, and Liu et al., (1987) J. Immunol., 139:3521-3526. As well, antibodies or other molecules that target the following tumor cell antigens could serve as appropriate targeting agents according to the invention: Apo2, CD20, CD40, muc-1, prostate specific membrane antigen (PSMA), prostate stem cell antigen (PSCA), epithelial growth factor receptor (EGFR), CD33, CD19, decay accelerating factor (DAF), EpCAM, CD52, carcinoembryonic antigen (CEA), TAG72 antigen, c-MET, or six-transmembrane epithelial antigen of the prostate (STEAP).

The caspases of the invention can be linked to the targeting molecule by any means known in the art to produce the caspase conjugate of the invention. For example, the caspase can be linked to the targeting molecule by covalent linkage. Methods of making covalent linkages are well known in the art and include methods such as the use of the heterobifunctional crosslinking reagent, SPDP (N-succinimidyl-3-(2-pyridyldithio)propionate) or SMCC (succinimidyl 4-(N-maleimidomethyl) cyclohexane-1-carboxylate [see, e.g., P. F. Thorpe et al., “The Preparation And Cytotoxic Properties Of Antibody-Toxin Conjugates,” Immunological Rev., 62, pp. 119-58 (1982); J. M. Lambert et al., supra, at p. 12038; G. F. Rowland et al., supra, at pp. 183-84 and J. Gallego et al., supra, at pp. 737-38].

More selective linkage can be achieved by using a heterobifunctional linker such as a maleimide-hydroxysuccinimide ester. Reaction of the latter with an enzyme will derivatize amine groups on the enzyme, and the derivative can then be reacted with, e.g., an antibody Fab fragment with free sulfhydryl groups (or a larger fragment or intact immunoglobulin with sulfhydryl groups appended thereto by, e.g., Traut's Reagent).

Preferred disulfide linkages are described in Arpicco et al., (1997) Bioconj. Chem. 8:327-337 and Dosio et al., (1998) Bioconj. Chem. 9:372-381.

It is advantageous to link the enzyme to a site on the targeting molecule such as an antibody, remote from the antigen binding site. This can be accomplished by, e.g., linkage to cleaved interchain sulfhydryl groups, as noted above. Another method involves reacting an antibody whose carbohydrate portion has been oxidized, with an enzyme which has at least one free amine function. This results in an initial Schiff base (imine) linkage, which is preferably stabilized by reduction to a secondary amine, e.g., by borohydride reduction, to form the final conjugate.

For antibody molecules and the like, conjugates comprising at least the antigen binding region of an antibody linked to at least a functionally active portion of a caspase of the invention can be constructed using recombinant DNA techniques well known in the art. Depending on the type of linkage, the caspase may be joined via its N- or C-terminus to the N- or C-terminus of a targeting molecule. For example, nucleic acid encoding a caspase may be operably linked to nucleic acid encoding the targeting molecule sequence, optionally via a linker domain. Typically the construct encodes a fusion protein comprising a targeting domain such as an antibody or antibody fragment wherein the N or C-terminus of the caspase is joined to the N-terminus of the antibody or antibody fragment. However, fusions where, for example, the C or N-terminus of the caspase is joined to the N or C-terminus of the targeting domain are also possible.

Preferred targeting domains are antibodies and antibody fragments. Typically, in such fusions the encoded fusion protein will retain at least CH1 and hinge domains, and in certain embodiments the CH2 and CH3 domains of the constant region of an immunoglobulin heavy chain. Fusions are also made, for example, to the C-terminus of the Fc portion of a constant domain, or immediately N-terminal to the CH1 of the heavy chain or the corresponding region of the light chain.

The precise amino acid site at which the fusion of the caspase to the immunoglobulin domain is made is not critical; particular sites are well known and may be selected in order to optimize the biological activity, secretion, or binding characteristics.

Because of the size of the conjugate, it will normally be preferably to link one antibody to one enzyme molecule. However, it may be advantageous to bind a plurality of antibody fragments, e.g., Fab or F(ab′)2 fragments, to a single enzyme to increase its binding affinity or efficiency to the antigen target. Alternatively, if the enzyme is not too bulky, it may be useful to link a plurality of enzyme molecules to a single antibody or antibody fragment to increase the turnover number of the conjugate and enhance the rate of deposition of the diagnostic or therapeutic agent at the target site. Conjugates of more than one caspase and antibody can also be used, provided they can reach the target site and they do not clear too fast. Mixtures of different sized conjugates, or conjugates that contain aggregates can be used, again with the same caveats just noted.

The targeting molecule-caspase conjugate can be further labeled with, or conjugated or adapted for conjugation to, a radioisotope or magnetic resonance image enhancing agent, to monitor its clearance from the circulatory system of the mammal and make certain that it has sufficiently localized at the target site, prior to the administration of the pro-agent. Alternatively, the conjugate can be tagged with a label, e.g., a radiolabel, a fluorescent label or the like, that permits its detection and quantitation in body fluids, e.g., blood and urine, so that targeting and/or clearance can be measured and/or inferred.

Any conventional method of radiolabeling which is suitable for labeling proteins for in vivo use will be generally suitable for labeling targeting agent/caspase conjugates, and often also for labeling substrate-agent conjugates, as will be noted below. This can be achieved by direct labeling with, e.g., I-131, I-123, metallation with, e.g., Tc-99m or Cu ions or the like, by conventional techniques, or by attaching a chelator for a radiometal or paramagnetic ion. Such chelators and their modes of attachment to antibodies are well known to the ordinary skilled artisan and are disclosed inter alia in, e.g., the aforementioned Goldenberg patents and in Childs et al., J. Nuc. Med., 26:293 (1985).

Drug Component

Appropriate drugs for use within the context of the present invention include any of those indicated in the course of treatment of a particular disease or disorder. Those skilled in the art will readily ascertain which molecules are appropriate for a given application by using one or more conventional means. For example, cytotoxic or chemotherapeutic agents are appropriate for in various cancer treatment protocols and may only be useful when administered as a proagent that is converted to a more active agent at a particular site. Examples of chemotherapeutic agents include Maytansinoids such as Maytansine and Ansamitocins, as well as synthetic analogs thereof, the Enediyne antibiotics including; Calicheamicins, in particular Calicheamicin γ₁^{I} and Calicheamicin θ₁^{I} (see, Angew, (1994) Chem. Int. Ed. Engl., 33:183-186), Dynemicins, in particular Dynemicin A and synthetic analogs thereof and Neocarzinostatin chromophore and related Chromoprotein enediyne antibiotic chromophores, Esperamicins (see U.S. Pat. No. 4,675,187) such as Esperamicin A₁; Adriamycin (Doxorubicin) and Morpholino-doxorubicin (Morpholino-ADR), Cyanomorpholino-doxorubicin (Cyanomorpholino-ADR), 2-Pyrrolino-Doxorubicin also known as AN-201, Deoxydoxorubicin, Tichothecenes, in particular T-2 Toxin, Verracurin A, Roridin A and Anguidine, Epothilones, Rhizoxin, Acetogenins, in particular Bullatacin and Bullatacinone, Cryptophycins, in particular Cryptophycin 1 and Cryptophycin 8, Dolastatin, Callystatin, CC-1065 and synthetic analogs, in particular Adozelesin, Carzelesin and Bizelesin, Duocarmycins and synthetic analogs, in particular KW-2189 and CBI-TMI, Sarcodictyins, Eleutherobin, Spongistatins, Bryostatins, Pancratistatin, Camptothecin and synthetic analogs, in particular Topotecan, Epirubicin, 5-Fluorouracil, Cytosine Arabinoside (“Ara-C”), Cyclophosphamide, Thiotepa, Busulfan, Taxoids, e.g. Paclitaxel (TAXOL®, Bristol-Myers Squibb Oncology, Princeton, N.J.) and Docetaxel (Taxotere, Rhône-Poulenc Rorer, Antony, Rnace), Methotrexate, Cisplatin, Melphalan and other related nitrogen mustards, Vinblastine, Bleomycin, Etoposide, Ifosfamide, Mitomycins such as Mitomycin C, Mitoxantrone, Vincristine, Vinorelbine, Carboplatin, Teniposide, Daunomycin, Carminomycin, Aminopterin, Dactinomycin. Also included in this definition are hormonal agents that act to regulate or inhibit hormone action on tumors such as tamoxifen and onapristone.

Design of Prodrug Moiety

The invention includes novel prodrugs that comprise a caspase cleavable prodrug moiety. Therefore, according to the invention an active drug is administered in the form of a prodrug requiring the action of a caspase of the invention for optimal activity. In general, a drug is selected based upon the disease or disorder to be treated. A caspase cleavable prodrug moiety is attached to the drug. The attachment site varies depending upon the drug but will typically be at a point which is necessary for high functional potency. Attachment of the prodrug moiety will result in a less active or minimally active drug.

The prodrug moiety will generally comprise at least four amino acids and will have an Asp in the P1 position. Therefore a prodrug moiety of the general formula P4-P3-P2-Asp is preferred within the context of the present invention. The prodrug moiety will be chosen with regard to the particular caspase being utilized. Specificities of the ten known human caspases have been described. The skilled artisan will reference Thornberry et al., (1997) supra in the design and construction of the appropriate prodrug moiety. For example, prodrug moiety of the general formula Asp-Xaa-Xaa-Asp will be preferred for caspases 3, 7 and 2 with Asp-Glu-Val-Asp (SEQ ID NO:3) being preferred for caspase 3 and 7 and Asp-Glu-His-Asp (SEQ ID NO:4) being preferred for caspase 2.

Preferred prodrugs have the general formula:

X-S4-S3-S2-Asp-Drug or X-S4-S3-S2-Asp-linker-Drug wherein X is optionally absent or for example an acyl group such as an acetyl group, and -linker- is an optional linker domain as more fully described herein.

Linker Domains

According to the present invention, the linker domain, is any group of molecules that provides a spatial bridge between two or more active domains as described in more detail herein below. According to this aspect of the invention, active domains such as a chemotherapeutic agent and a caspase cleavable prodrug moiety are linked together, as for example by chemical conjugation. The linker component of the hybrid molecule of the invention does not necessarily participate in but may contribute to the function of the hybrid molecule. Therefore, according to the present invention, the linker domain, is any group of molecules that provides a spatial bridge between a prodrug moiety as, for example, a peptide domain and a drug domain.

The linker domain can be of variable length and makeup. The artisan will consider the length of the linker molecule and its makeup including plasma stability, its compatability with the caspase active site, the ability to be self-removed (Carl, Chakravarty and Katzenellenbogen (1981) J. Medicinal Chem. 24(5):479-480); its solubility and the ability of the modified drug to be taken up by the cells. The linker domain preferably allows for the peptide domain of the hybrid molecule to interact, substantially free of spacial/conformational restrictions to the coordinant caspase molecule. Therefore, the length of the linker domain is dependent upon the character of the two functional domains, e.g., the peptide and the drug domains of the hybrid molecule. Appropriate linker domains are constructed keeping in mind that preferred linker domains provide an unstable linkage in the absence of the caspase cleavable prodrug moiety to the parent drug such that upon cleavage of the prodrug the linker is rapidly lost to liberate free active parent drug. Preferred linker domains therefore are “self-immolative.” A preferred linker domain is described in Dubowchik et al., (1998) Bioorg. Med. Chem. Letts. 8:3341-3346 and Dubowchik et al., (1998) Bioorg. Med. Chem. Letts. 8:3347-3352.

Chemical Synthesis

One method of producing the compounds of the invention involves chemical synthesis. This can be accomplished by using methodologies well known in the art (see Kelley, R. F. & Winkler, M. E. in Genetic Engineering Principles and Methods, Setlow, J. K, ed., Plenum Press, N.Y., vol. 12, pp 1-19 (1990), Stewart, J. M. Young, J. D., Solid Phase Peptide Synthesis, Pierce Chemical Co., Rockford, Ill. (1984); see also U.S. Pat. Nos. 4,105,603; 3,972,859; 3,842,067; and 3,862,925).

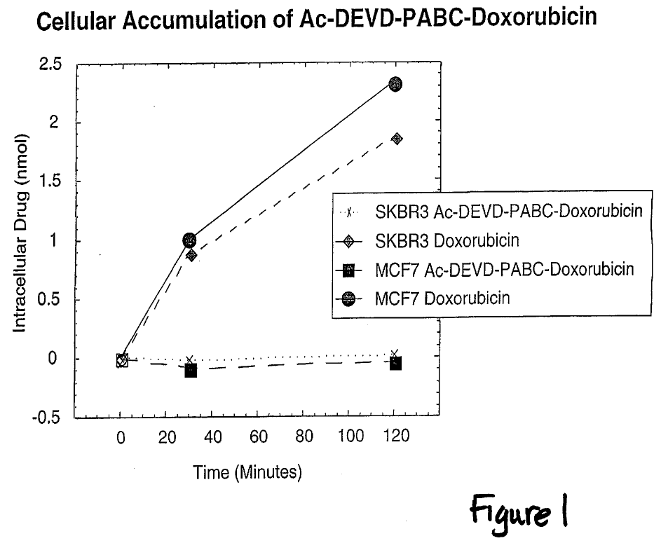

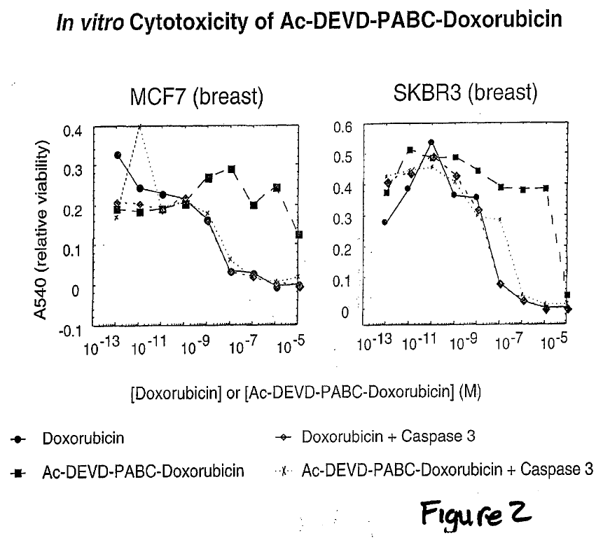

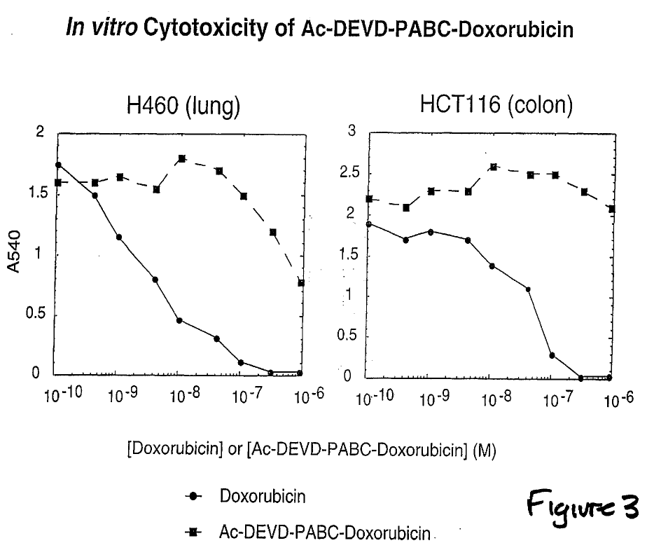

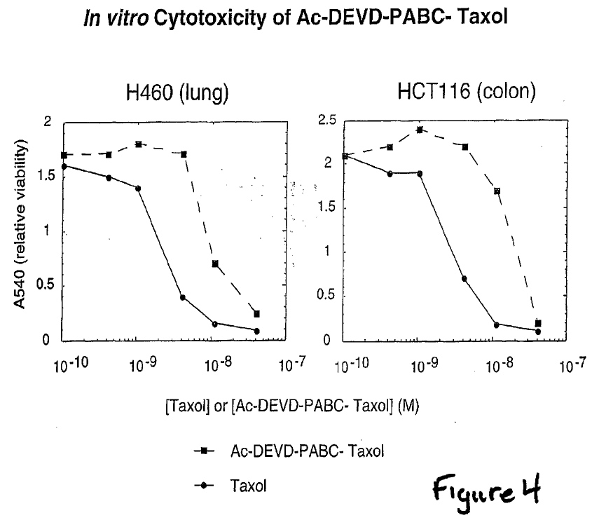

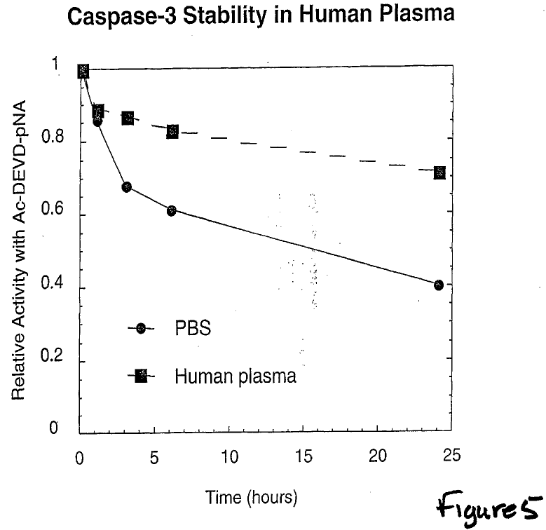

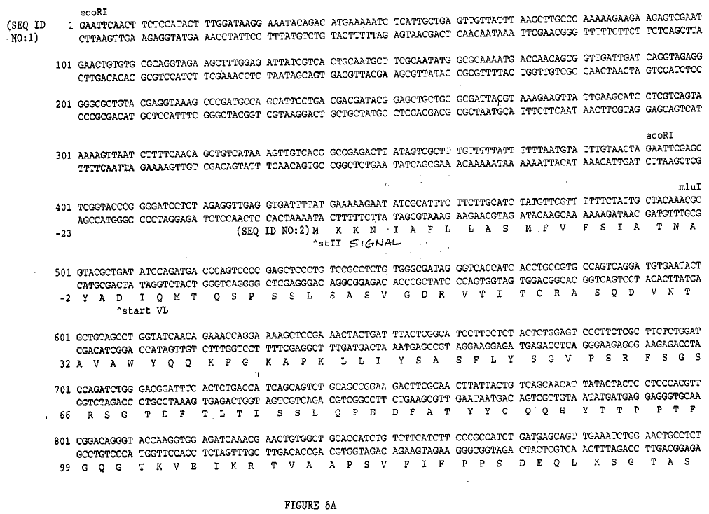

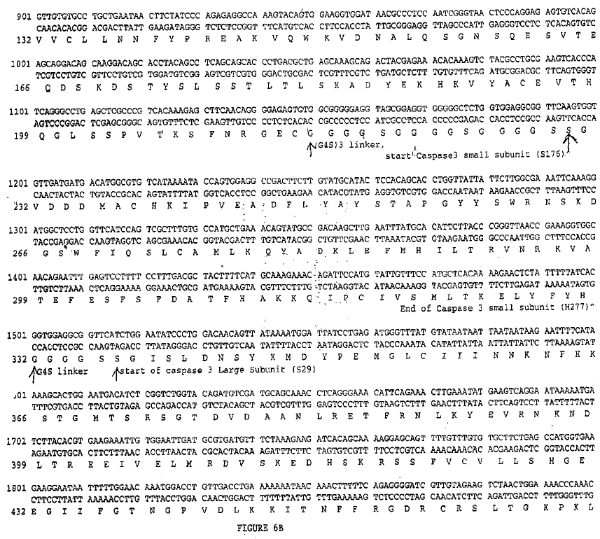

Proagents of the invention can be conveniently prepared using a combination solid phase peptide synthesis (Merrifield, (1964) J. Am. Chem. Soc., 85:2149; Houghten, (1985) Proc. Natl. Acad. Sci. USA, 82:5132 an organic chemical or recombinant synthesis. Solid phase synthesis begins at the carboxy terminus of the putative peptide by coupling a protected amino acid to an inert solid support. The inert solid support can be any macromolecule capable of serving as an anchor for the C-terminus of the initial amino acid. Typically, the macromolecular support is a cross-linked polymeric resin (e.g. a polyamide or polystyrene resin) as shown in FIGS. 1-1 and 1-2, on pages 2 and 4 of Stewart and Young, supra. In one embodiment, the C-terminal amino acid is coupled to a polystyrene resin to form a benzylic ester. A macromolecular support is selected such that the peptide anchor link is stable under the conditions used to deprotect the α-amino group of the blocked amino acids in peptide synthesis. If a base-labile α-protecting group is used, then it is desirable to use an acid-labile link between the peptide and the solid support. For example, an acid-labile ether resin is effective for base-labile Fmoc-amino acid peptide synthesis as described on page 16 of Stewart and Young, supra. Alternatively, a peptide anchor link and α-protecting group that are differentially labile to acidolysis can be used. For example, an aminomethyl resin such as the phenylacetamidomethyl (Pam) resin works well in conjunction with Boc-amino acid peptide synthesis as described on pages 11-12 of Stewart and Young, supra.

After the initial amino acid is coupled to an inert solid support, the α-amino protecting group of the initial amino acid is removed with, for example, trifluoroacetic acid (TFA) in methylene chloride and neutralizing in, for example, triethylamine (TEA). Following deprotection of the initial amino acid's (α-amino group, the next at-amino and sidechain protected amino acid in the synthesis is added. The remaining α-amino and, if necessary, side chain protected amino acids are then coupled sequentially in the desired order by condensation to obtain an intermediate compound connected to the solid support. Alternatively, some amino acids may be coupled to one another to form a fragment of the desired peptide followed by addition of the peptide fragment to the growing solid phase peptide chain.

The condensation reaction between two amino acids, or an amino acid and a peptide, or a peptide and a peptide can be carried out according to the usual condensation methods such as the azide method, mixed acid anhydride method, DCC (N,N′-dicyclohexylcarbodiimide) or DIC (N,N′-diisopropylcarbodiimide) methods, active ester method, p-nitrophenyl ester method, BOP (benzotriazole-1-yl-oxy-tris [dimethylamino] phosphonium hexafluorophosphate) method, N-hydroxysuccinic acid imido ester method, etc, Woodward reagent K method, HBTU (O-[benzotriazol-1-yl]-1,1,3,3-tetramethyluronium hexafluorophosphate) method, HATU (O-[7-azabenzotriazol-1-yl]-1,1,3,3-tetramethyluronium hexafluorophosphate) method, and PyBOP (benzotriazol-1-yl-oxy-trispyrrolidinophosphonium hexafluorophosphate) method.

It is common in the chemical synthesis of peptides to protect any reactive side-chain groups of the amino acids with suitable protecting groups. Ultimately, these protecting groups are removed after the desired polypeptide chain has been sequentially assembled. Also common is the protection of the α-amino group on an amino acid or peptide fragment while the C-terminal carboxy group of the amino acid or peptide fragment reacts with the free N-terminal amino group of the growing solid phase polypeptide chain, followed by the selective removal of the α-amino group to permit the addition of the next amino acid or peptide fragment to the solid phase polypeptide chain. Accordingly, it is common in polypeptide synthesis that an intermediate compound is produced which contains each of the amino acid residues located in the desired sequence in the peptide chain wherein individual residues still carry side-chain protecting groups. These protecting groups can be removed substantially at the same time to produce the desired polypeptide product following removal from the solid phase.

α- and ε-amino side chains can be protected with benzyloxycarbonyl (abbreviated Z), isonicotinyloxycarbonyl (iNOC), o-chlorobenzyloxycarbonyl [Z(2Cl)], p-nitrobenzyloxycarbonyl [Z(NO2)], p-methoxybenzyloxycarbonyl [Z(OMe)], t-butoxycarbonyl (Boc), t-amyloxycarbonyl (Aoc), isobornyloxycarbonyl, adamantyloxycarbonyl, 2-(4-biphenyl)-2-propyloxycarbonyl (Bpoc), 9-fluorenylmethoxycarbonyl (Fmoc), methylsulfonyethoxycarbonyl (Msc), trifluoroacetyl, phthalyl, formyl, 2-nitrophenylsulphenyl (NPS), diphenylphosphinothioyl (Ppt), and diphenylphosphinothioyl (Mpt) groups, and the like.

Protective groups for the carboxy functional group are exemplified by benzyl ester (OBzl), cyclohexyl ester (OChx), 4-nitrobenzyl ester (ONb), t-butyl ester (O^{t}-Bu), 4-pyridylmethyl ester (Opic), allyl ester (OAll), and the like. It is often desirable that specific amino acids such as arginine, cysteine, and serine possessing a functional group other than amino and carboxyl groups are protected by a suitable protective group. For example, the guanidino group of arginine may be protected with nitro, p-toluenesulfonyl, benzyloxycarbonyl, adamantyloxycarbonyl, p-methoxybenzesulfonyl, 4-methoxy-2,6-dimethylbenzenesulfonyl (Nds), 1,3,5-trimethylphenysulfonyl (Mts), and the like. The thiol group of cysteine can be protected with p-methoxybenzyl, trityl, and the like.

Many of the blocked amino acids described above can be obtained from commercial sources such as Novabiochem (San Diego, Calif.), Bachem CA (Torrence, Calif.) or Peninsula Labs (Belmont, Calif.).

Stewart and Young, supra, provides detailed information regarding procedures for preparing peptides. Protection of α-amino groups is described on pages 14-18, and side chain blockage is described on pages 18-28. A table of protecting groups for amine, hydroxyl and sulfhydryl functions is provided on pages 149-151.

After the desired amino acid sequence has been completed, the peptide can be cleaved away from the solid support, recovered and purified. The peptide is removed from the solid support by a reagent capable of disrupting the peptide-solid phase link, and optionally deprotects blocked side chain functional groups on the peptide. In one embodiment, the peptide is cleaved away from the solid phase by acidolysis with liquid hydrofluoric acid (HF), which also removes any remaining side chain protective groups. Preferably, in order to avoid alkylation of residues in the peptide (for example, alkylation of methionine, cysteine, and tyrosine residues), the acidolysis reaction mixture contains thio-cresol and cresol scavengers. Following HF cleavage, the resin is washed with ether, and the free peptide is extracted from the solid phase with sequential washes of acetic acid solutions. The combined washes are lyophilized, and the peptide is purified.

Recombinant Synthesis

The present invention encompasses a composition of matter comprising isolated nucleic acid, preferably DNA, encoding a caspase conjugate described herein. DNAs encoding the conjugates of the invention can be prepared by a variety of methods known in the art. These methods include, but are not limited to, chemical synthesis by any of the methods described in Engels et al., (1989) Agnew. Chem. Int. Ed. Engl., 28:716-734, the entire disclosure of which is incorporated herein by reference, such as the triester, phosphite, phosphoramidite and H-phosphonate methods. In one embodiment, codons preferred by the expression host cell are used in the design of the encoding DNA. Alternatively, DNA encoding the conjugate can be altered to encode one or more variants by using recombinant DNA techniques, such as site specific mutagenesis (Kunkel et al., (1991) Methods Enzymol. 204:125-139; Carter, P., et al., (1986) Nucl. Acids. Res. 13:4331; Zoller, M. J. et al., (1982) Nucl. Acids Res. 10:6487), cassette mutagenesis (Wells, J. A., et al., (1985) Gene 34:315), restriction selection mutagenesis (Wells, J. A., et al., (1986) Philos. Trans, R. Soc. London SerA 317, 415), and the like.

The invention further comprises an expression control sequence operably linked to the DNA molecule encoding a conjugate of the invention, and an expression vector, such as a plasmid, comprising the DNA molecule, wherein the control sequence is recognized by a host cell transformed with the vector. In general, plasmid vectors contain replication and control sequences which are derived from species compatible with the host cell. The vector ordinarily carries a replication site, as well as sequences which encode proteins that are capable of providing phenotypic selection in transformed cells.

Suitable host cells for expressing the DNA include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635).

In addition to prokaryotes, eukaryotic organisms, such as yeasts, or cells derived from multicellular organisms can be used as host cells. For expression in yeast host cells, such as common baker's yeast or Saccharomyces cerevisiae, suitable vectors include episomally replicating vectors based on the 2-micron plasmid, integration vectors, and yeast artificial chromosome (YAC) vectors. Suitable host cells for expression also are derived from multicellular organisms. Examples of invertebrate cells include insect cells such as Drosophila S2 and Spodoptera Sf9, as well as plant cells. For expression in insect host cells, such as Sf9 cells, suitable vectors include baculoviral vectors. For expression in plant host cells, particularly dicotyledonous plant hosts, such as tobacco, suitable expression vectors include vectors derived from the Ti plasmid of Agrobacterium tumefaciens.

Examples of useful mammalian host cells include monkey kidney CV1 tine transformed by SV40 (COS-7, ATCC CRL 1651); human embryonic kidney line (293 or 293 cells subcloned for growth in suspension culture, Graham et al., (1977) J. Gen Virol., 36:59); baby hamster kidney cells (BHK, ATCC CCL 10); Chinese hamster ovary cells/-DHFR (CHO, Urlaub and Chasin, (1980) Proc. Natl. Acad. Sci. USA, 77:4216); mouse sertoli cells (TM4, Mather, (1980) Biol. Reprod., 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., (1982) Annals N.Y. Acad. Sci., 383:44-68); MRC 5 cells; FS4 cells; and a human hepatoma cell line (Hep G2).

For expression in prokaryotic hosts, suitable vectors include pBR322 (ATCC No. 37,017), phGH107 (ATCC No. 40,011), pBO475, pS0132, pRIT5, any vector in the pRIT20or pRIT30 series (Nilsson and Abrahmsen, (1990) Meth. Enzymol., 185:144-161), pRIT2T, pKK233-2, pDR540 and pPL-lambda. Prokaryotic host cells containing the expression vectors of the present invention include E. coli K12 strain 294 (ATCC NO. 31446), E coli strain JM101 (Messing et al., (1981) Nucl. Acid Res., 9:309), E. coli strain B, E. coli strain 1776 (ATCC No. 31537), E. coli c600 (Appleyard, Genetics, 39: 440 (1954)), E. coli W3110 (F-, gamma-, prototrophic, ATCC No. 27325), E. coli strain 27C7 (W3110, tonA, phoA E15, (argF-lac)169, ptr3, degP41, ompT. kanr) (U.S. Pat. No. 5,288,931, ATCC No. 55,244), Bacillus subtilis, Salmonella typhimurium, Serratia marcesans, and Pseudomonas species.

For expression in mammalian host cells, useful vectors include vectors derived from SV40, vectors derived from cytomegalovirus such as the pRK vectors, including pRK5 and pRK7 (Suva et al., (1987) Science, 237:893-896; EP 307,247 (Mar. 15, 1989), EP 278,776 (Aug. 17, 1988)) vectors derived from vaccinia viruses or other pox viruses, and retroviral vectors such as vectors derived from Moloney's murine leukemia virus (MoMLV).

Optionally, the DNA encoding the conjugate of interest is operably linked to a secretory leader sequence resulting in secretion of the expression product by the host cell into the culture medium. Examples of secretory leader sequences include stII, ecotin, lamB, herpes GD, lpp, alkaline phosphatase, invertase, and alpha factor. Also suitable for use herein is the 36 amino acid leader sequence of protein A (Abrahmsen et al., (1985) EMBO J., 4:3901).

Host cells are transfected and preferably transformed with the above-described expression or cloning vectors of this invention and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO4 precipitation and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in section 1.82 of Sambrook et al., Molecular Cloning (2nd ed.), Cold Spring Harbor Laboratory, NY (1989), is generally used for prokaryotes or other cells that contain substantial cell-wall barriers. Infection with Agrobacterium tumefaciens is used for transformation of certain plant cells, as described by Shaw et al., (1983) Gene, 23:315 and WO 89/05859 published 29 Jun. 1989. For mammalian cells without such cell walls, the calcium phosphate precipitation method described in sections 16.30-16.37 of Sambrook et al., supra, is preferred. General aspects of mammalian cell host system transformations have been described by Axel in U.S. Pat. No. 4,399,216 issued 16 Aug. 1983. Transformations into yeast are typically carried out according to the method of Van Solingen et al., (1977) J. Bact., 130:946 and Hsiao et al., (1979) Proc. Natl. Acad. Sci. (USA), 76:3829. However, other methods for introducing DNA into cells such as by nuclear injection, electroporation, or by protoplast fusion may also be used.

Therapeutic Protocols

The method of the invention is normally practiced by parenteral injection. The various types of parenteral injections can be, but are not limited to intracavitary (e.g., intraperitoneal), intravenous, intraarterial, intrapleural, intrathecal, intramuscular, intralymphatic and regional intraarterial, intralesional, subcutaneous, catheter perfusion and the like.

For cancer imaging and/or therapy, intravenous, intraarterial or intrapleural administration is normally used for lung, breast, and leukemic tumors. Intraperitoneal administration is advantageous for ovarian tumors. Intrathecal administration is advantageous for brain tumors and leukemia. Subcutaneous administration is advantageous for Hodgkin's disease, lymphoma and breast carcinoma. Catheter perfusion is useful for metastatic lung, breast or germ cell carcinomas of the liver. Intralesional administration is useful for lung and breast lesions.

The above illustrates the general methods of administration of targeting agent-caspase conjugates according to the present invention. It will be appreciated that the modes of administration of the two different conjugates, i.e., the caspase conjugate and the prodrug, may not be the same, since the clearance pathways and biodistributions of the conjugates will generally differ. For example, intraperitoneal administration of an antibody-enzyme conjugate may be advantageous for targeting an ovarian tumor, whereas intravenous administration of a proagent conjugate may he desirable because of better control of the rate of deposit and ease of monitoring of the clearance rate.

The targeting agent-caspase conjugate will generally be administered as an aqueous solution in sterile vehicle suitable for in vivo administration. Advantageously, dosage units of about 50 micrograms to about 5 mg of the targeting agent-caspase conjugate will be administered, either in a single dose or in divided doses, although smaller or larger doses may be indicated in particular cases. It may be necessary to reduce the dosage and/or use antibodies from other species and/or hypoallergenic antibodies, e.g., fragments or hybrid human or primate antibodies, to reduce patient sensitivity, especially for therapy and especially if repeated administrations are indicated for a therapy course or for additional diagnostic procedures.

It usually takes from about 2 to 14 days for IgG antibody to localize at the target site and substantially clear from the circulatory system of the mammal prior to administration of the pro-agent conjugate. The corresponding localization and clearance time for F(ab′)2 antibody fragments is from about 2 to 7 days, and from about 1 to 3 days for Fab and Fab′ antibody fragments. Other antibodies may require different time frames to localize at the target site, and the above time frames may be affected by the presence of the conjugated enzyme. Again, it is noted that labeling the antibody-enzyme conjugate permits monitoring of localization and clearance.

IgG is normally metabolized in the liver and, to a lesser extent, in the digestive system. F(ab′)2 are normally metabolized primarily in the kidney, but can also be metabolized in the liver and the digestive system. Fab and Fab′ are normally metabolized primarily in the kidney, but can also be metabolized in the liver and the digestive system.

Normally, it will be necessary for at least about 0.0001% of the injected dose of antibody-enzyme conjugate to localize at the target site prior to administration of the substrate-agent conjugate. To the extent that a higher targeting efficiency for this conjugate is achieved, this percentage can be greater, and a reduced dosage can be administered.

It follows that an effective amount of an antibody-enzyme conjugate is that amount sufficient to target the conjugate to the antigen at the target site and thereby bind an amount of the enzyme sufficient to transform enough of the soluble substrate-agent conjugate to product to result in accretion of an effective diagnostic or therapeutic amount of the agent at the target site.

The substrate-therapeutic or diagnostic agent conjugate will be generally administered as an aqueous solution in PBS. Again, this will be a sterile solution if intended for human use. The substrate-agent conjugate will be administered after a sufficient time has passed for the antibody-enzyme conjugate to localized at the target site and substantially clear from the circulatory system of the mammal.

Pharmaceutical Compositions

Pharmaceutical compositions of the compounds of the invention are prepared for storage by mixing a caspase conjugate or prodrug containing compound having the desired degree of purity with optional pharmaceutically acceptable carriers, excipients or stabilizers (Remington's Pharmaceutical Sciences 16th edition, Osol, A. Ed. 119801), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The following examples are offered by way of illustration and not by way of limitation. The disclosures of all citations in the specification are expressly incorporated herein by reference.

### EXAMPLES

### Example I

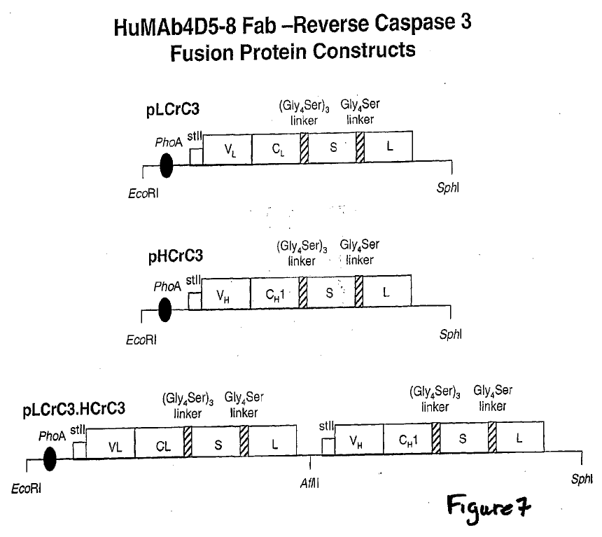

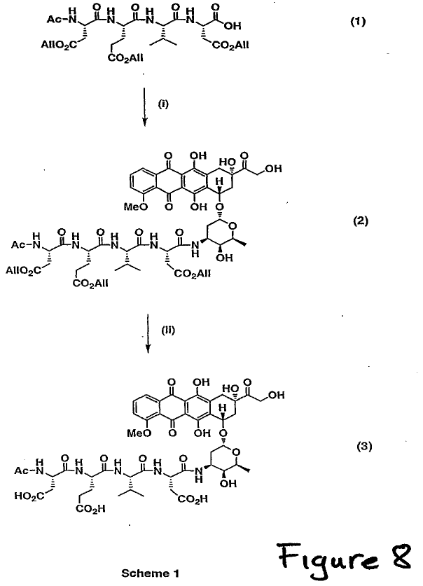

### Preparation of Ac-DEVD-doxorubicin (FIG. 8)

Procedures:

(i) A solution of peptide [1] (38 μmol), 1,3-dicyclohexylcarbodiimide (40 μmol) and N-hydroxysuccinimide (57 μmol) in anhydrous DMF (1.5 ml) at 0° C. was treated with ethyldiisopropylamine (98 μmol) for 10 min. A solution of Doxorubicin hydrochloride (32 μmol) and ethyldiisopropylamine (98 μmol) in anhydrous DMF (3.0 ml) was added dropwise, and the mixture was allowed to warm to 23° C. for 72 h, protected from light. Concentration in vacuo and purification of the residue by preparative HPLC yielded [2] as an orange-red amorphous solid (8.9 μmol, 28%).

[HPLC: C-18 reverse-phase 21 mm i.d.×250 mm column; flow-rate 10 ml/min.; 40-60% (acetonitrile+0.1% TFA) in (water+0.1% TFA) linear gradient elution over 60 min.; retention time 28 min.]

(ii) A solution of [2] (4.7 μmol) and tetrakis(triphenylphosphine)palladium (0) (0.3 μmol) in degassed, anhydrous DMF (1.5 ml) at 23° C. was treated with acetic acid (70 μmol) and tributyltin hydride (41 μmol) and stirred while protected from light. The mixture was treated with further quantities of with acetic acid (87 μmol) and tributyltin hydride (45 μmol) at 1.5 h, and with tetrakis(triphenylphosphine)palladium (0) (0.3 μmol) at 34 h and at 72 h (0.6 μmol). Concentration in vacuo after 91 h and purification of the residue by preparative HPLC yielded [3] as an orange-red amorphous solid (2.1 μmol, 44%).

[HPLC: 0-60% linear gradient elution over 60 min., other conditions as before; retention time 43 min.]

### Example II

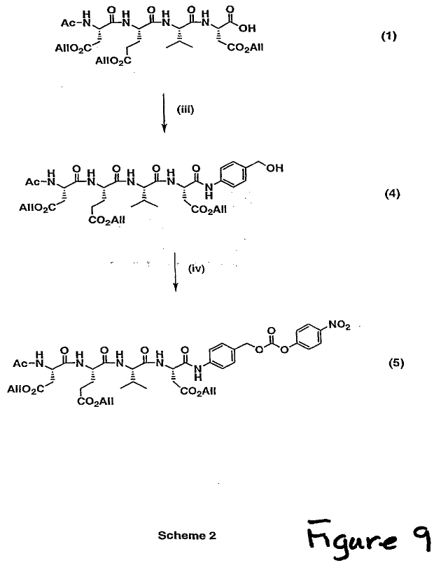

### Preparation of Ac-DEVD-PABC Prodrug Moiety (FIG. 9)

Procedure:

(iii) A solution of the peptide [1] (88 μmol), 4-aminobenzyl alcohol (179 μmol) and 2-ethoxy-1-ethoxycarbonyl-1,2-dihydroquinoline (178 μmol) in anhydrous DMF (1.0 ml) was allowed to react at 23° C. for 24 h. Concentration in vacuo and purification of the residue by preparative HPLC yielded [4] as a white amorphous solid (63 μmol, 72%).

[HPLC: 0-60% linear gradient elution over 60 min., other conditions as before; retention time 48 min.]

(iv) To the peptide [4] (181 μmol) and 4-nitrophenyl chloroformate (216 μmol) in anhydrous dichloromethane (6.0 ml) at 23° C. was added 2,6-lutidine (541 μmol). After 2 h the mixture was diluted with anhydrous DMF (2.0 ml) and treated with a second portion of 2,6-lutidine (360 μmol). Further quantities of 2,6-lutidine (860 μmol) and 4-nitrophenyl chloroformate (175 μmol) were added at 24 h, 27 h and at 46 h. After 84 h the mixture was treated with saturated aqueous sodium bicarbonate and extracted three times with ethyl acetate (total 150 ml). The combined organic phases were washed with aqueous citric acid (80 ml, 0.5 M), saturated aqueous sodium bicarbonate solution and brine, dried over anhydrous sodium sulfate and concentrated in vacuo. Purification of the residue by preparative HPLC yielded [5] as a white amorphous solid (131 μmol, 72%).

[HPLC: Elution 0-40% over 15 min., 40-60% over 45 min., other conditions as before; retention time 46 min.]

### Example III

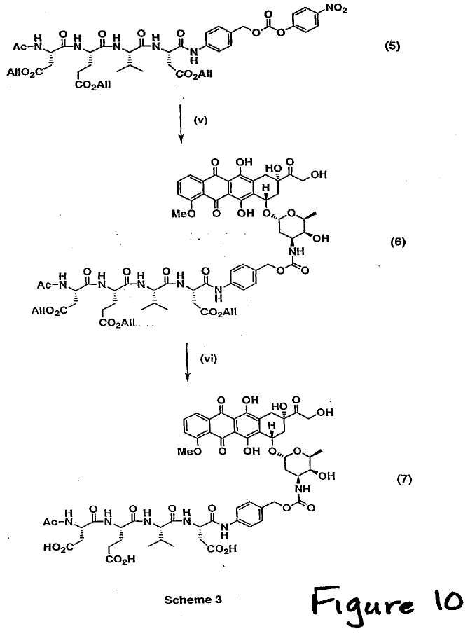

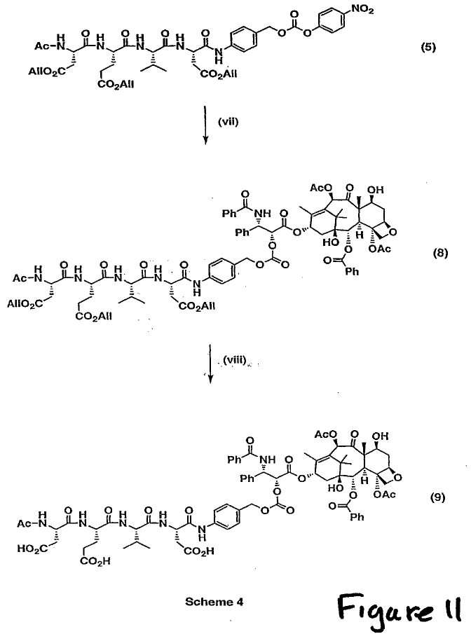

### Preparation of Ac-DEVD-PABC-doxorubicin (FIG. 10)

Procedure:

(v) A solution of the carbonate [5] (74 μmol) and Doxorubicin hydrochloride (86 μmol) in anhydrous DMF (10 ml) at 23° C. was treated dropwise with ethyldiisopropylamine (402 μmol) and stirred for 16 h, protected from light. Concentration in vacuo and purification of the residue by preparative HPLC yielded [6] as an orange-red amorphous solid (45 μmol, 61%).

[HPLC: Elution 0-40% over 15 min., 40-60% over 45 min., other conditions as before; retention time 45 min.]

(vi) To a solution of [6] (12 μmol) and tetrakis(triphenylphosphine)palladium (0) (1.5 μmol) in degassed anhydrous DMF (2.0 ml) at 23° C. was added acetic acid (245 μmol) and tributyltin hydride (123 μmol). The mixture was stirred for 16 h while protected from light, and then concentrated in vacuo. Purification of the residue by preparative HPLC yielded [7] as a deep orange-red amorphous solid (5.7 μmol, 47%).

[HPLC: 0-50% linear gradient elution over 60 min., other conditions as before; retention time 52 min.; repurified using isocratic elution at 30%; retention time 35 min.]

### Example IV

### Preparation of Ac-DEVD-PABC-paclitaxel (FIG. 10)

Procedure:

(vii) A solution of the carbonate [5] (57 μmol), Paclitaxel (58 μmol) and 4-dimethylaminopyridine (176 μmol) in anhydrous acetonitrile (10 ml) was allowed to react at 23° C. for 20 h. Concentration in vacuo and purification of the residue by preparative HPLC yielded [8] as a white amorphous solid (47 μmol, 83%).

[HPLC: Elution 0-50% over 15 min., 50-70% over 45 min., other conditions as before; retention time 38 min.]

(viii) To a solution of [8] (46 μmol) and tetrakis(triphenylphosphine)palladium (0) (5.1 μmol) in degassed, anhydrous DMF (6.0 ml) at 23° C. was added acetic acid (926 μmol) and tributyltin hydride (457 μmol). The mixture was stirred for 18 h while protected from light, and then concentrated in vacuo. Purification of the residue by preparative HPLC yielded [9] as a deep orange-red amorphous solid (31 μmol, 68%).

[HPLC: Elution 0-40% over 15 min., 40-60% over 45 min., other conditions as before; retention time 34 min.]

Abbreviations:

Ac=Acetyl
All=Allyl (2-Propen-1-yl)
Bu=n-Butyl
DCC=1,3-Dicyclohexylcarbodiimide
DCM=Dichloromethane
DIPEA=Diisopropylethylamine
DMAP=4-(Dimethylamino)pyridine
DMF=N,N-Dimethylformamide
EEDQ=2-Ethoxy-1-ethoxycarbonyl-1,2-dihydroquinoline
HPLC=High-performance liquid chromatography
Me=Methyl
Ph=Phenyl
Su=N-Succinimidyl
TFA=Trifluoroacetic acid

### Example V

### Cellular Accumulation of Doxorubicin and Ac-DEVD-PABC-Doxorubicin

SK-BR-3 and MCF7 breast carcinoma cells (American Type Culture Collection (ATCC), Rockville, Md.) were cultured in Dulbecco's modified Eagle's medium:Ham's nutrient F-12 (50:50) supplemented with 2 μM glutamine, 100 units/mL penicillin, 100 μg/L streptomycin (Gibco BRL, Grand Island, N.Y.), and 10% (w/v) bovine fetal serum (Hyclone, Logan, Utah) (cell media) at 37° C., 5% CO₂. Adherantly growing cells were detached by treatment with phosphate-buffered saline containing 0.05% trypsin, 0.6 mM EDTA (5 min) and then resuspended at 10⁶ cells per mL in fresh cell media. Cells were either used directly (“untreated”) or supplemented with doxorubicin or Ac-DEVD-PABC-doxorubicin into a final concentration of 10 μM. Cells were incubated for 0 to 2 h at 37° C. and then pelleted by centrifugation (5 min, 500 g, 4° C.). The supernatant was discarded and the cells then gently resuspended in 10 mL ice-cold phosphate-buffered saline. The cell pelleting and resuspension steps were then repeated. Doxorubicin (12.5 nmol, 1.25 nmol or 0.125 nmol) was added to the previously untreated cells for use in preparing a standard curve. The cell pellets were dissolved in 200 μL 0.3 M HCl in 50% (v/v) ethanol and transferred to 1.5 mL Eppendorf tubes. Debris was pelleted in a microcentrifuge (5 min, 14 000 rpm, 25° C.). 150 μL of supernatant was then transferred to a well of a 96 well plate. Fluorescence measurements were then undertaken using a fluorescent plate reader (Fluoroskan, Helsinki, Finland) with absorption and emission wavelengths of 485 and 590 nm respectively. Uptake of doxorubicin was estimated from a standard curve prepared using known quantities of doxorubicin that were added to the previously untreated cells. Doxorubicin was found to significantly accumulate inside MCF7 and SKBR3 cells whereas Ac-DEVD-PABC-doxorubicin did not (FIG. 1).

### Example VI

### Prodrug Cytotoxicity Assay 1

SK-BR-3 and MCF7 breast carcinoma cells (ATCC) were cultured in Dulbecco's modified Eagle's medium:Ham's nutrient F-12 (50:50) supplemented with 2 mM glutamine, 100 units/mL penicillin, 100 μg/mL streptomycin (Gibco BRL), and 10% (w/v) bovine fetal serum (Hyclone) (cell media) at 37° C., 5% CO₂. Cells were seeded at 10,000 cells/well (SK-BR-3), or 3,000 cells/well (MCF7) in 96-well tissue culture plates (Falcon, Becton-Dickinson, Franklin Lakes, N.J.) and allowed to attach for 24 h. Cell media was aspirated, and replaced with fresh cell media (100 μL/well) containing 2 mM PIPES, 1 mM DTT, 0.1 mM EDTA, 0.01% CHAPS, 10 mM NaCl and 1% sucrose in the presence of 0 to 10⁴ M Ac-DEVD-PABC-doxorubicin or doxorubicin and in the presence or absence of 13 ng recombinant human caspase 3 (Calbiochem, San Diego, Calif.). After 3 h incubation plates were washed twice with cell media (37° C.), and further incubated for a total assay length of 120 h. The assay was terminated by staining with 0-25% (w/v) crystal violet in 50% (v/v) ethanol. The plates were then rinsed with water and the remaining crystal violet solubilized using 50 mM sodium citrate (pH 4.5) in 50% (v/v) ethanol. The absorbance was read at 540 nm using a microtiter plate reader (SpectraMax 340, Molecular Devices, Sunnyvale, Calif.).

### Example VII

### Activation of Ac-DEVD-PABC-Doxorubicin by Caspase 3

Ac-DEVD-PABC-doxorubicin (60 μM) was incubated with 1 ng recombinant human caspase 3 (Calbiochem) in the presence or absence of the caspase 3 inhibitor, Z-DEVD-FMK (400 μM) (Calbiochem) in phosphate-buffered saline containing 5% (v/v) dimethyl sulfoxide and 45 mM DTT in a total reaction volume of 700 mL. A control reaction was performed in which caspase 3 and inhibitor were omitted. Reactions were incubated for 0 to 2 h at 37° C. and then frozen in dry ice. Reactions were then analyzed by reverse phase HPLC using a Microsorb-MV C18 reverse-phase column (4.6 mm internal diameter×250 mm length, 5 μm particle size, 100 Å pore size) (Rainin, Emeryville, Calif.) under isocratic conditions: 0.1% (v/v) TFA acid, 35% (v/v) acetonitrile at a flow rate of 1.5 mL/min whilst monitoring the absorbance at 254 nm. The retention times for Ac-DEVD-PABC-doxorubicin and doxorubicin were 7.7 min and 5.1 min respectively. AC-DEVD-PABC-doxorubicin was found to be more than 100-fold less toxic than doxorubicin against MCF7 and SK-BR-3 cells. Ac-DEVD-PABC-doxorubicin wan equally toxic to doxorubicin following treatment with caspase 3 (FIG. 2). Ac-DEVD-PABC-doxorubicin is efficiently activated by caspase 3 as shown by the conversion to doxorubicin (Table II).

### Example VIII

### Activation of Ac-DEVD-PABC-Taxol by Caspase 3

Ac-DEVD-PABC-taxol (35 μM) was incubated with 1 ng recombinant human caspase 3 (Calbiochem) in the presence or absence of the caspase 3 inhibitor, Z-DEVD-FMK (400 μM) (Calbiochem) in phosphate-buffered saline containing 5% (v/v) dimethyl sulfoxide and 45 mM DTT in a total reaction volume of 700 μL. A control reaction was performed in which caspase 3 and inhibitor were omitted. Reactions were incubated for 0 to 2 h at 37° C. and then frozen in dry ice. Reactions were then analyzed by reverse phase HPLC using a Microsorb-MV C18 reverse-phase column (4.6 mm internal diameter×250 mm length, 5 μm particle size, 100 Å pore size) (Rainin) under isocratic conditions. 0.1% (v/v) TFA, 46% (v/v) acetonitrile at a now rate of 1.5 mL/min whilst monitoring the absorbance at 254 nm. The retention times for taxol and Ac-DEVD-PABC-taxol were 13.3 min 10.4 min, respectively. AcDEVD-PABC-taxol is efficiently activated by caspase 3 as shown by the conversion to taxol (Table III).

### Example IX

### Prodrug Cytotoxicity Assay 2

Human lung carcinoma cells (H460, SK-MES-1), colon carcinoma cells (HCT116), breast carcinoma cell lines (BT-474, MCF7, SK-BR-3) and normal lung fibroblasts (WI-38) were purchased from the ATCC and maintained in high glucose DMEM:Ham's F-12 (50:50) supplemented with 10% (v/v) heat-inactivated FBS (Gibco BRL) and 2 mM 1-glutamine. Normal human mammary epithelial cells (HMEC) were purchased from Clonetics/Biowhittaker (Walkersville, Md.) and maintained in mammary epithelial growth media (MEGM, Clonetics). Cells were detached from culture flasks by treating with phosphate-buffered saline containing 0.05% (w/v) trypsin and 0.6 mM EDTA (5 min) and seeded into 96-well microtiter plates at densities of 10⁴ cells per well (WI-38 and HMEC), 1.5×10⁴ per well (H460, SK-MES-1 and HCT116) or 2×10⁴ cells per well (MCF7, BT-474, SK-BR-3). After allowing the cells to attach overnight, drugs or prodrugs were added at the following final concentrations: doxorubicin or Ac-DEVD-PABC-doxorubicin, 0 to 1 μM; taxol or Ac-DEVD-PABC-taxol, 0 to 0.04 μM. Following 72 h treatment, media were gently removed from the wells and the cell monolayers were stained with 0.5% (w/v) crystal violet dye in 20% (v/v) methanol. The plates were rinsed extensively with water and allowed to dry. The dye was then solubilized with 50 mM sodium citrate buffer, pH 4.2, in 50% (v/v) ethanol (200 μL per well), the plates were agitated for 30 min at 25° C. and the absorbance read at 540 nm using a 340 ATC microtiter plate reader (SLT LabInstruments, Salzburg, Austria).

### Example X

### Plasma Stability of Caspase 3

Fresh heparin-treated blood was centrifuged to pellet cells and platelets (5 min, 1500 g, 4° C.). The supernatant (plasma) was respun in a microcentrifuge (5 min, 14 000 rpm, 25° C.). Recombinant caspase 3 (500 ng) was added to either 200 μL plasma or 200 μL phosphate-buffered saline. Aliquots were removed after 0 to 24 h incubation at 37° C., flash frozen in liquid nitrogen and stored at −70° C. Plasma samples were thawed and diluted 5-25 fold in caspase buffer (20 mM PIPES, 10 mM DTT, 1 mM EDTA, 0.1% CHAPS, 10% sucrose, 100 mM NaCl, pH 7.2) containing 75 μg/mL of the chromogenic substrate, acetyl-L-Asp-L-Glu-L-Val-L-Asp-p-nitroanilide (Calbiochem). Substrate hydrolysis was monitored by following the change in absorbance at 410 nm at 25° C. a microtiter plate reader (SpectraMax 340, Molecular Devices).

### Example XI

### Construction of Plasmids Encoding Antibody Fragment Fusion Proteins with Reverse Caspase 3

1) Description of Plasmids

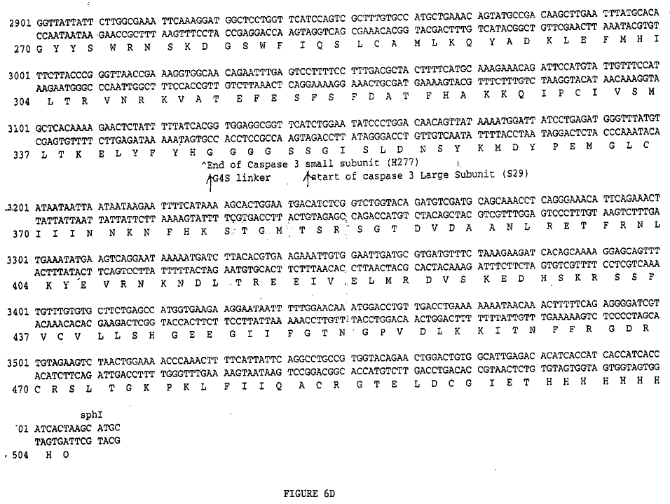

The plasmid, pLCrC3, encodes the light chain of HuMab4D5-8 Fab (Carter et al., 1992a supra; Carter et al., 1992b, Bio/Technology 10: 163-167) fused via a linker encoding (Gly₄Ser)₃ to a gene encoding a constitutively active form of caspase 3 known as reverse caspase 3 (Srinivasula et al., 1998 supra) (shown schematically in FIG. 7).

The plasmid, pHCrC3, encodes the heavy chain Fd fragment of HuMab4D5-8 Fab (Carter et al., 1992a,b supra) fused via a linker encoding (Gly₄Ser)₃ to a gene encoding reverse caspase 3 (Srinivasula et al., 1998 supra) (shown schematically in FIG. 7).

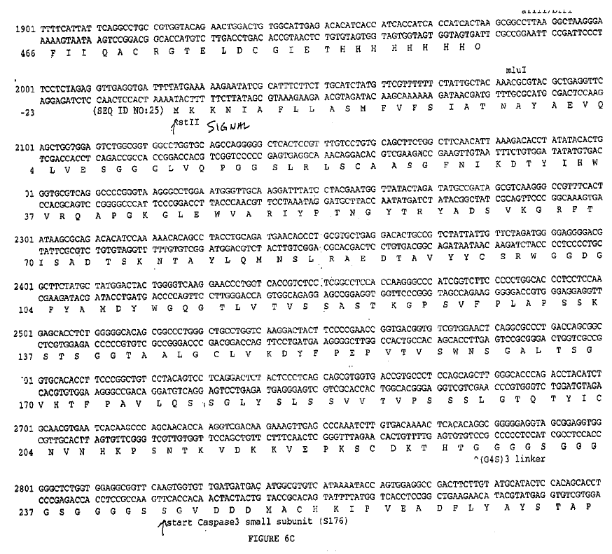

The plasmid (pLCrC3.HCrC3) contains genes encoding the light chain and heavy chains Fd fragments of HuMab4D5-8 Fab (Carter et al., 1992a,b supra) each fused via a linker encoding (Gly₄Ser)₃ to a gene encoding a constitutively active form of caspase 3 known as reverse caspase 3 (Srinivasula et al., 1998 supra). The biscistronic operon in pLCrC3.HCrC3 encoding HuMAb4D5-8 Fab-reverse caspase 3 is shown in schematic form in FIG. 7 and as annotated DNA and protein sequences in FIG. 6. The operon is under the trancriptional control of the phoA promoter (C. W. Chang et al. (1986) Gene 44:121-125) inducible by phosphate starvation. The humanized variable domains (V_{L} and V_{H}) of huMAb4D5-8 are precisely fused on their 5′ ends to a gene segment encoding the heat stable enterotoxin II (stII) signal sequence (R N Picken et al. (1983) Infect. Immun. 42:269-275) to direct secretion of the polypeptide to the periplasmic space of E. coli. Each copy of reverse caspase 3 is followed by a sequence encoding 8 histidines to facilitate purification of the resultant fusion protein by immobilized metal affinity chromatography.

The plasmid pLCrC3.HCrC3s differs from pLCrC3.HCrC3 in that codons 214 and 223 in huMAb4D5-8 light chain and heavy chain Fd fragment, respectively, encode serine residues rather than cysteine residues.

The plasmid pET21b.rC3 contains a gene encoding reverse caspase 3 (Srinivasalu et al., (1998) supra) in the vector pET21b (Novagen, Madison, Wis.).

Construction of Plasmid pLCrC3

Plasmid, pLCrC3, was assembled by recombinant PCR (Rashtenian (1995) Curr. Opin. Biotech. 6:30-36) starting from plasmids pAK19 (Carter et al. (1992a,b) supra) encoding the Fab′ fragment of HuMab4D5-8 and plasmid pET21b.rC3 encoding reverse caspase 3 in pET21b (Novagen, Madison, Wis.). The gene encoding the light chain of HuMab4D5-8 was first PCR-amplified from plasmid pAK19 using the primers:

(SEQ ID NO: 14)

P1: 5′GCTACAAACGCGTACGCTGATATCCAGATGACCCAGTCCCCGAG
CTCCCTG 3′

(SEQ ID NO:15)

P2: 5′CCCCCACCTCCGCTACCTCCCCCGCCACACTCTCCCCTGTTGAA
GCTCTTTGTGACG 3′

Similarly, the gene encoding reverse caspase 3 was PCR-amplified from pET21b.rC3 using the primers:

(SEQ ID NO:16)

P3: 5′CGGGGGAGGTAGCGGAGGTGGGGGCTCTGGTGGAGGCGGTTCAA
GTGGTGTTGATG 3′,

(SEQ ID NO:17)

P4: 5′GCCGTCGCATGCTTAGTGATGGTGATGGTGATGGTGATGTGTCT
CAATGCCACAGTC 3′.

The PCR reaction conditions (“PCR 1 conditions”) were as follows: 50-100 ng DNA template in 20 mM Tris-HCl (pH 8.8), 10 mM KCl, 10 mM (NH₄)₂SO₄, 2 mM MgSO_{4, 0.1}% Triton X-100, 0.1 mg/mL bovine serum albumin (BSA), 200 μM of each dNTP, 25 μmol of each primer, 2.5 U PfuTurbo (Stratagene, La Jolla, Calif.) in a total volume 50 μL. Thermocycling conditions (“thermocycling 1 conditions”) were as follows: 95° C. for 5 min followed by 30 cycles of 95° C. for 20 s, 55° C. for 20 s, 72° C. for 90 s, then finally one cycle of 72° C. for 10 min.

These PCR products were gel purified on a 1% agarose gel (Gibco BRL). Bands of the appropriate molecular weight (˜690 bp and ˜840 bp respectively) were excised and DNA extracted using a QIAquick Gel extraction kit (Qiagen, Valencia, Calif.). Second, these 2 DNA fragments were then mixed at a 1:1 ratio and subjected to a second round of PCR using the primers P1 and P4 using PCR 1 conditions and the following thermocycling conditions (“thermocycling 2 conditions”): 95° C. for 5 min followed by 30 cycles of 95° C. for 20 s, 50° C. for 20 s, 72° C. for 90 s, then finally one cycle of 72° C. for 10 min. The PCR product was cloned into pAK19 using the MluI and SphI sites to create pLCrC3, and then verified by dideoxynucleotide sequencing.

Construction of Plasmid pHCrC3

Plasmid, pHCrC3, was assembled by recombinant PCR (A. Rashtchian (1995) supra) starting from plasmid pAK19 encoding the Fab′ fragment of HuMab4D5-8 and plasmid pET21b.rC3. The gene encoding the heavy chain of HuMab4D5-8 was first PCR-amplified from plasmid pAK19 using the primers:

(SEQ ID NO: 18)

P5 5′TGCTACAAACGCGTACGCTGAGGTTCAGCTGGTGGAGTCTGGCGG
TGGCCTG 3′

(SEQ ID NO: 19)

P6 5′CCCCACCTCCGCTACCTCCCCCGCCTGTGTGAGTTTTGTCACAAG
ATTTGGGC 3′

Similarly, the gene encoding reverse caspase 3 was PCR-amplified from pET21b.rC3 using the primers:

(SEQ ID NO:16)

P3: 5′CGGGGGAGGTAGCGGAGGTGGGGGCTCTGGTGGAGGCGGTTCAA
GTGGTGTTGATG 3′,

(SEQ ID NO:17)

P4: 5′GCCGTCGCATGCTTAGTGATGGTGATGGTGATGGTGATGTGTCT
CAATGCCACAGTC 3′.

PCR 1 Conditions and Thermocycling 1 Conditions

These PCR products were gel purified on a 1% agarose gel (Gibco BRL). Bands of the appropriate molecular weight (˜730 bp and ˜840 bp respectively) were excised and DNA extracted using a QIAquick Gel extraction kit (Qiagen). Next, these 2 DNA fragments were mixed at a 1:1 ratio and subjected to a second round of PCR using the primers P5 and P4 under PCR 1 conditions and thermocycling 2 conditions. The PCR product was cloned into pAK19 using the MluI and SphI sites to create pHCrC3, and then verified by dideoxynucleotide sequencing.

Construction of Plasmid pLCrC3.HCrC3

Plasmid pLCrC3.HCrC3 was created by ligation of 3 DNA fragments: ˜4914 bp MluI/SphI fragment from pAK19, ˜1489 bp MluI/AflII PCR fragment from pLCrC3 and ˜1623 bp AflII/SphI PCR fragment from pHCrC3.

The MluI/AflII fragment from pLCrC3 was created by PCR amplification using primers: P7: 5′GCTACAAACGCGTACGCTGATATCCAGATGACCCAGTCCCCGAGCTCCCTG 3′ (SEQ ID NO:14) and P1 under PCR 1 conditions and thermocycling 1 conditions followed by digestion with MluI and AflII. Similarly, the AflII/SphI fragment from pHCrC3 was created by PCR amplification using primers P4 and P8, 5′TAAGCGGCCTTAAGGCTAAGGGATCCTCTAGAGGTTGAGGTGATTTTATG 3′ (SEQ ID NO:20) under PCR 1 conditions and thermocycling 1 conditions followed by digestion with AflII and SphI. Plasmid pLCrC3.HCrC3 was verified by dideoxynucleotide sequencing.

Construction of Plasmid pLCrC3.HCrC3s

Plasmid pLCrC3.HCrC3s was created from pLCrC3.HCrC3 by mutating the codons at position 214 and 223 in huMAb4D5-8 light chain and heavy chain Fd fragment, respectively, so that they encode serine residues rather than cysteine residues. Sequential mutagenesis of light and heavy chains was accomplished using a QuikChange site-directed mutagenesis kit (Stratagene). The light chain mutations encoding C214S were accomplished using the 2 synthetic DNA fragments: P9 5′ CTTCAACAGGGGAGAGTCTGGCGGG 3′ (SEQ ID NO:21) and P10 5′ CCCGCCAGACTCTCCCCTGTFGAAG 3′ (SEQ ID NO:22), whereas the heavy chain mutations encoding C223S were accomplished using the 2 synthetic DNA fragments, P11 5′GCCCAAATCTTCTGACAAAACTCAC 3′ (SEQ ID NO:23), and P12 5′GTGAGTTTTGTCAGAAGATTTGGGC 3′ (SEQ ID NO:24).

### Example XII

### Shake Flask Expression of huMAb4D5-8 Fab-Reverse Caspase 3 Fusion Proteins

Plasmids pLCrC3.HCrC3 and pLCrC3.HCrC3s were transformed into E. coli strain 25F2 (Carter et al., (1992b) supra) and grown in 5 mL of Luria-Bertani (LB) broth containing 50 μg/mL carbenecillin rotating overnight at 37° C. One mL of these overnight cultures was used to inoculated 250 mL complete CRAP medium containing 50 μg/mL carbenecillin and grown overnight with shaking at 30° C. (Complete CRAP medium is prepared as follows: 3.57 g (NH₄)₂SO₄, 0.71 g NaCitrate-2H₂O, 1.07 g KCl, 5.36 g yeast extract, 5.36 g Hycase SF-Sheffield, adjust pH with KOH to 7.3 and volume to 872 mL with deionized water. Autoclave and then cool to 55° C. Add 110 mL 1 M MOPS pH 7.3, 11 mL 50% glucose, 7.0 mL 1 M MgSO₄). Cells were pelleted by centrifugation (3000 g, 15 min, 4° C.) and then resuspended in 25 mL 10 mM Tris-HCl pH 7.6, 1 mM EDTA. Samples were gently agitated at 30 min at 4° C. and then centrifuged (27 000 g, 20 min, 4° C.). The supernatants (“schockates”) were then adjusted to 100 mM sodium phosphate (pH 8.0), 300 mM NaCl, 20 mM imidazole, 10 mM MgCl₂ and 10 mM β-mercaptoethanol. The fusion proteins were then purified by immobilized metal affinity chromatography (IMAC) using Ni-NTA superflow agarose (Qiagen). Bound protein was eluted with 1 mL 100 mM sodium phosphate (pH 8.0), containing 300 mM NaCl, 250 mM imidazole and 10 mM β-mercaptoethanol. “Shockates” and IMAC purified samples were analyzed by quantitative anti-HER2 Fab ELISA, anti-polyhistidine ELISA and assayed for reverse caspase 3 activity using the chromogenic substrate. Acetyl-L-Asp-L-Glu-L-Val-L-Asp-P-nitroanilide

### Example XIII

### Quantitative Anti-HER2 Fab ELISA

96-well ELISA plates (Maxisorp, Nunc) were coated (16 h, 4° C.) with 100 μL per well of 1 μg/mL HER2 extracellular domain in Na₂CO₃ (pH 9.6). The plates were washed with PBST (0.05% Tween 20 in phosphate-buffered saline) using a plate-washer (Skanwasher 300, Skatron Instruments) and then blocked with 280 μL PBST containing 3% skimmed milk (Carnation) (PBST-SM) (1 h, 25° C.). The plates were washed twice with PBST then incubated with a dilution series of samples and standards in PBST-SM (1 h, 25° C.). The standard used was huMAb4D5-8 Fab (Carter et al. (1992a,b) supra), (R. F. Kelley et al. (1992) Biochemistry 31:5434-5441) serially 2-fold diluted over the range 1-400 ng/mL. The plates were washed with PBST and then incubated with an anti-human κ light chain-horse-raddish peroxidase conjugate (Catalog # 55233, ICN Pharmaceuticals, Aurora, Ohio): 100 μL per well of 1:5000 dilution of conjugate in PBST-SM. The plates were washed and then incubated with 100 μL per well of freshly mixed TMB substrates (Kirkegaard and Perry Laboratories, Gaithersburg, Md.) (2-15 min, 25° C.). The reaction was quenched by the addition of 100 μL per well of 1 M phosphoric acid. The absorbance at 450 nm minus that at 650 nm was measured using a microtiter plate reader (SpectraMax 340, Molecular Devices). The data were corrected for background and then subjected to a non-linear least squares (Kaleidagraph version 3.0.5, Synergy Software, Reading, Pa.): A₄₅₀−A₆₅₀=(c*A)/(c+B), where c is the concentration of standard, A and B are constants. The calculated fit was used to estimate the concentration of huMAb4D5-8 Fab-reverse caspase 3 fusion protein in the samples.

### Example XIV

### Anti Polyhistidine ELISA

96-well ELISA plates (Maxisorp, Nunc) were coated (16 h, 4° C.) with 100 μL per well of 1 μg/mL HER2 extracellular domain in Na₂CO₃ (pH 9.6). The plates were washed with PBST (0.05% Tween 20 in phosphate-buffered saline) using a plate-washer (Skanwasher 300, Skatron Instruments) and then blocked with 280 μL PBST containing 3% skimmed milk (Carnation) (PBST-SM) (1 h, 25° C.). The plates were washed twice with PBST then incubated with a dilution series of samples and positive control in ELISA assay buffer (phosphate-buffered saline containing 0.5% (w/v) bovine serum albumin, and 0.01% thimerosal) (1 h, 25° C.). The positive control used was huMAb4D5-8 (Carter et al. (1992a) supra) scFv fragment with a His₆ tag serially 2-fold diluted over the range 1-400 ng/mL. The plates were washed with PBST and then incubated with biotin-labeled penta-His antibody (Qiagen): 100 μL per well of 1:5000 dilution of antibody in ELISA assay buffer for (1 h, 25° C.). The plates were washed and then incubate with a streptavidin-horse raddish peroxidase conjugate; 100 μL per well of 1:5000 dilution of conjugate in ELISA assay buffer (1 h, 25° C.). The plates were washed and then incubated with 100 μL per well of freshly mixed TMB substrates (Kirkegaard and Perry Laboratories) (2-15 min, 25° C.). The reaction was quenched by the addition of 100 μL per well of 1 M phosphoric acid. The absorbance at 450 nm minus that at 650 nm was measured using a microtiter plate reader (SpectraMax 340, Molecular Devices).

### Example XV

Reverse Caspase 3 Activity Assay

Samples and separately recombinant human caspase 3, (Calbiochem) were serially 2-fold diluted in caspase buffer (20 mM PIPES, 10 mM DTT, 1 mM EDTA, 0.1% CHAPS, 10% sucrose, 100 mM NaCl, pH 7.2) in 96-well ELISA plates. The highest concentration of caspase 3 standard used was 125 ng per well. The final assay volume was 250 μl caspase buffer containing 250 μM chromogenic substrate, acetyl-L-Asp-L-Glu-L-Val-L-Asp-p-nitroanilide (Calbiochem). The absorbance at 405 nm was measured every 30 s for 30 min using a microtiter plate reader (SpectraMax 340, Molecular Devices).

Analysis of Ac-DEVD-PABC-Doxorubicin Cleavage

TABLE II

| | | Incubation | Conver- |
|---|---|---|---|
| Sample | Additions | Time (min) | sion* (%) |
| Ac-DEVD-PABC-Doxorubicin | Caspase 3 | 0 | 1.8 |
| Ac-DEVD-PABC-Doxorubicin | Caspase 3 | 30 | 17 |
| Ac-DEVD-PABC-Doxorubicin | Caspase 3 | 120 | 76 |
| Ac-DEVD-PABC-Doxorubicin | Caspase 3 + | 0 | 0 |
| | inhibitor | | |
| Ac-DEVD-PABC-Doxorubicin | Caspase 3 + | 30 0 | |
| | inhibitor | | |
| Ac-DEVD-PABC-Doxorubicin | Caspase 3 + | 120 | 0 |
| | inhibitor | | |

$Conversion = \frac{Doxorubicin peak area \times 100}{\begin{matrix}( Doxorubicin peak area + \\ Ac \text{-} DEVD \text{-} PABC \text{-} Doxorubicin peak area )\end{matrix}} .$
Peak areas were not normalised.

Analysis of Ac-DEVD-PABC-Taxol Cleavage

TABLE III

| | | Incubation | Conversion* |
|---|---|---|---|
| Sample | Additions | Time (min) | (%) |
| Ac-DEVD-PABC-taxol | Caspase 3 | 0 | 0 |
| Ac-DEVD-PABC-taxol | Caspase 3 | 30 | 65 |
| Ac-DEVD-PABC-taxol | Caspase 3 | 120 | 100 |
| Ac-DEVD-PABC-taxol | Caspase 3 + | 0 | 0 |
| | inhibitor | | |
| Ac-DEVD-PABC-taxol | Caspase 3 + | 30 | 0 |
| | inhibitor | | |
| Ac-DEVD-PABC-taxol | Caspase 3 + | 120 | 0 |
| | inhibitor | | |

$Conversion = \frac{Taxol peak area \times 100}{\begin{matrix}( Taxol peak area + \\ Ac \text{-} DEVD \text{-} PABC \text{-} Taxol peak are )\end{matrix}}$
Peak areas were not normalised.

Characterization of huMAb4D-8 Fab-Reverse Caspase Fusion Protein

The expression titer of huMAb4D-8 Fab-reverse caspase 3 fusion protein following propagation of pLCrC3.HCrC3 and pLCrC3.HCrC3s in E. coli 25F2 was ˜200 ng/mL and ˜0.6 ng/mL as estimated by quantitative anti-HER2 Fab ELISA of corresponding shockates. In both cases the presence of Fab and reverse caspase within the same molecule was confirmed by qualitative anti-polyhistidine ELISA. These two ELISA assays also confirm that the Fab fragment is functional for binding to HER2. The function of the reverse caspase 3 was confirmed by demonstrating that it is capable of hydrolyzing the chromogenic substrate acetyl-L-Asp-L-Glu-L-Val-L-Asp-p-nitroanilide.
1. Carter, P., et al., High level Escherichia coli expression and production of a bivalent humanized antibody fragment. Bio/Technology, 1992. 10(2): p. 163-7.
2. Srinivasula, S. M., et al., Generation of constitutively active recombinant caspases-3 and -6 by rearrangement of their subunits. Journal of Biological Chemistry, 1998. 273(17): p. 10107-11.
3. Carter, P., et al., Humanization of an anti-p185HER2 antibody for human cancer therapy. Proceedings of the National Academy of Sciences of the United States of America, 1992. 89(10): p. 4285-9.
4. Kelley, R. F., et al., Antigen binding thermodynamics and antiproliferative effects of chimeric and humanized anti-p185HER2 antibody Fab fragments. Biochemistry, 1992. 31(24): p. 5434-41. antibody Fab fragments. Biochemistry, 1992. 31(24): p. 5434-41.

What is claimed is:

1. A method for the delivery of an active agent to a cell type of interest comprising the steps of;
a) administering an effective amount of a cell type targeted conjugate comprising a caspase which converts a caspase convertable pro-agent to an active agent and
b) administrating a caspase convertable pro-agent.

2. The method of claim 1 wherein the caspase is a mammalian caspase.

3. The method of claim 2 wherein the caspase is a human caspase.

4. The method of claim 3 wherein the caspase is a proapoptotic caspase.

5. The method of claim 4 wherein the caspase is selected from the group consisting of caspase 2, caspase 3 and caspase 7.

6. The method of claim 5 wherein the caspase is caspase 3.

7. The method of claim 1 wherein the cell type of interest is a tumor cell.

8. The method of claim 1 wherein the cell type targeted conjugate is an antibody conjugate.

9. The method of claim 8 wherein the antibody is a polyclonal antibody.

10. The method of claim 8 wherein the antibody is a monoclonal antibody.

11. The method of claim 8 wherein the antibody is an antibody fragment.

12. The method of claim 11 wherein the antibody fragment is a F(ab′)₂.

13. The method of claim 1 wherein the active agent is a cytotoxic agent.

14. The method of claim 13 wherein the cytotoxic agent is selected from the group consisting of doxorubicin, daunorubicin, epirubicin, taxol, taxotere, vincristine, vinblastine, mitomycin C, etoposide, methotrexate, cisplatin, clyclophosphamide, mephalan, Halotestin, clyclophosphamide, Thio-TEPA, chlorambucil, 5-FU, and cytoxan.

15. The method of claim 14 wherein the cytotxic agent is doxorubicin.

16. A pharmaceutical composition which comprises a cell type targeted conjugate of a caspase.

17. A pharmaceutical composition which comprises antibody conjugated caspase.

18. A prodrug comprsing a caspase cleavable prodrug moiety.

19. The prodrug of claim 18 wherein the prodrug moiety has the sequence Asp-Xaa-Xaa-Asp.

20. The prodrug of claim 19 wherein the prodrug moiety has the sequence Asp-Glu-Val-Asp.

21. The prodrug of claim 20 comprising doxorubicin.

22. The prodrug of claim 20 comprising paclitaxel.

23. A kit comprising an antibody conjugated caspase.

24. The kit of claim 23 further comprising a pro-agent which is converted to a more active agent by the antibody conjugated caspase.

25. A method of treating a mammal comprising the step of administering to the mammal a therapeutically effective amount of an pro-agent which is converted to an active agent by a caspase.

26. A method of treating a mammal comprising the steps of administering to the mammal a therapeutically effective amount of a pro-agent which is converted to an active agent by a caspase and a cell type targeted caspase.


SEQUENCE LISTING (25 sequences)

SEQ ID NO: 1 - DNA, 3614 bp, Homo sapiens
gaattcaacttctccatactttggataaggaaatacagacatgaaaaatctcattgctgagttgttatttaagcttgcccaaaaagaagaagagtcgaatgaactgtgtgcgcaggtagaagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtaactagaattcgagctcggtacccggggatcctctagaggttgaggtgattttatgaaaaagaatatcgcatttcttcttgcatctatgttcgttttttctattgctacaaacgcgtacgctgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaccatcagcagtctgcagccggaagacttcgcaacttattactgtcagcaacattatactactcctcccacgttcggacagggtaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcgggggaggtagcggaggtgggggctctggtggaggcggttcaagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcacggtggaggcggttcatctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacacatcaccatcaccatcaccatcactaagcggccttaaggctaagggatcctctagaggttgaggtgattttatgaaaaagaatatcgcatttcttcttgcatctatgttcgttttttctattgctacaaacgcgtacgctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacaggcgggggaggtagcggaggtgggggctctggtggaggcggttcaagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcacggtggaggcggttcatctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacacatcaccatcaccatcaccatcactaagcatgc

SEQ ID NO: 2 - protein, 513 aa, Homo sapiens
MKKNIAFLLASMFVFSIATNAYADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHGGGGSSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETHHHHHHHH

SEQ ID NO: 3 - protein, 4 aa, Artificial Sequence
  Act_Site
DEVD

SEQ ID NO: 4 - protein, 4 aa, Artificial Sequence
  Act_Site
DEID

SEQ ID NO: 5 - protein, 5 aa, Artificial Sequence
  Conserved Caspase Active Site Motif
  MISC_FEATURE (4)..(4): Xaa = Arg, Gln or Gly
QACXG

SEQ ID NO: 6 - protein, 4 aa, Artificial Sequence
  Group I Caspase Optimal Sequence
WEHD

SEQ ID NO: 7 - protein, 4 aa, Artificial Sequence
  Caspase 9 Optimal Sequence
LEHD

SEQ ID NO: 8 - protein, 4 aa, Artificial Sequence
  Caspase 2 Optimal Sequence
DEHD

SEQ ID NO: 9 - protein, 4 aa, Artificial Sequence
  CED-3 Optimal Sequence
DETD

SEQ ID NO: 10 - protein, 4 aa, Artificial Sequence
  Caspase 6 Optimal Sequence
VEHD

SEQ ID NO: 11 - protein, 4 aa, Artificial Sequence
  Caspase 8 Optimal Sequence
LETD

SEQ ID NO: 12 - protein, 4 aa, Artificial Sequence
  Caspase 10 Substrate Sequence
  MISC_FEATURE (3)..(3): Xaa = Nle
LEXD

SEQ ID NO: 13 - protein, 4 aa, Artificial Sequence
  Granzyme B Optimal Sequence
IEPD

SEQ ID NO: 14 - DNA, 51 bp, Artificial Sequence
  Primer-bind
gctacaaacgcgtacgctgatatccagatgacccagtccccgagctccctg

SEQ ID NO: 15 - DNA, 57 bp, Artificial Sequence
  Primer-bind
cccccacctccgctacctcccccgccacactctcccctgttgaagctctttgtgacg

SEQ ID NO: 16 - DNA, 56 bp, Artificial Sequence
  Primer-bind
cgggggaggtagcggaggtgggggctctggtggaggcggttcaagtggtgttgatg

SEQ ID NO: 17 - DNA, 57 bp, Artificial Sequence
  Primer-bind
gccgtcgcatgcttagtgatggtgatggtgatggtgatgtgtctcaatgccacagtc

SEQ ID NO: 18 - DNA, 52 bp, Artificial Sequence
  Primer-bind
tgctacaaacgcgtacgctgaggttcagctggtggagtctggcggtggcctg

SEQ ID NO: 19 - DNA, 53 bp, Artificial Sequence
  Primer-bind
ccccacctccgctacctcccccgcctgtgtgagttttgtcacaagatttgggc

SEQ ID NO: 20 - DNA, 50 bp, Artificial Sequence
  Primer-bind
taagcggccttaaggctaagggatcctctagaggttgaggtgattttatg

SEQ ID NO: 21 - DNA, 25 bp, Artificial Sequence
  Primer-bind
cttcaacaggggagagtctggcggg

SEQ ID NO: 22 - DNA, 25 bp, Artificial Sequence
  Primer-bind
cccgccagactctcccctgttgaag

SEQ ID NO: 23 - DNA, 25 bp, Artificial Sequence
  Primer-bind
gcccaaatcttctgacaaaactcac

SEQ ID NO: 24 - DNA, 25 bp, Artificial Sequence
  Primer-bind
gtgagttttgtcagaagatttgggc

SEQ ID NO: 25 - protein, 527 aa, Homo sapiens
MKKNIAFLLASMFVFSIATNAYAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHGGGGSSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETHHHHHHHH